(19) United States
(12) Patent Application Publication
(10) Pub. No.: US 2022/0096515 A1
(43) Pub. Date: Mar. 31, 2022

(54) Methods and Formulations for Preventing Neurological or Psychiatric Disorders

(71) Applicant: Huntington Medical Research Institutes, Pasadena, CA (US)

(72) Inventor: Anju Vasudevan, Pasadena, CA (US)

(73) Assignee: Huntington Medical Research Institutes, Pasadena, CA (US)

(21) Appl. No.: 17/488,061

(22) Filed: Sep. 28, 2021

Related U.S. Application Data
(60) Provisional application No. 63/084,230, filed on Sep. 28, 2020.

(51) Int. Cl. A61K 31/7084; A61K 9/00

(52) U.S. Cl. CPC A61K 31/7084; A61K 9/0053

(57) ABSTRACT

Methods of treatment and pharmaceutical formulations configured to prevent psychiatric diseases and/or disorders in offspring are provided. The methods and treatments use a regulator of angiogenesis pathways. The regulator can restore GABA secretion and neuronal migration. The regulator may be administered during pregnancy of an individual, thus allowing proper development of a fetal brain.

### CROSS-REFERENCE TO RELATED APPLICATIONS

The current application claims priority to U.S. Provisional Patent Application No. 63/084,230, filed Sep. 28, 2020; the disclosure of which is hereby incorporated by reference in its entirety.

### GOVERNMENT SPONSORSHIP

This invention was made in part with government support under Grant Nos. 1R01NS100808-01A1 and 1R01MH110438-01awarded by the National Institutes of Health. The government has certain rights to this invention.

### FIELD OF THE DISCLOSURE

The present disclosure relates to methods of treatment and pharmaceutical formulations to treating and/or prevent psychiatric disorders in utero.

### BACKGROUND OF THE DISCLOSURE

The global burden of mental health disorders and their consequences have been steadily increasing. While some studies have established an autonomous link between blood vessels and the developmental roots of psychiatric disease, no treatment has been identified to prevent the onset of psychiatric symptoms and/or diseases. (See e.g., S. Li, et al., Endothelial cell-derived GABA signaling modulates neuronal migration and postnatal behavior. Cell research 28, 221 (February, 2018); C. Won, et al., Autonomous vascular networks synchronize GABA neuron migration in the embryonic forebrain. Nat Commun 4, 2149 (2013); S. Li, K. et al. Endothelial VEGF sculpts cortical cytoarchitecture. The Journal of neuroscience: the official journal of the Society for Neuroscience 33, 14809 (Sep. 11, 2013); A. Vasudevan, et al., Compartment-specific transcription factors orchestrate angiogenesis gradients in the embryonic brain. Nat Neurosci 11, 429 (April, 2008); and Y. K. Choi and A. Vasudevan, Mechanistic insights into autocrine and paracrine roles of endothelial GABA signaling in the embryonic forebrain. Scientific reports 9, 16256 (Nov. 7, 2019); the disclosures of which are incorporated herein by reference in their entireties.)

By investigating the importance of novel GABA related gene expression in embryonic forebrain endothelial cells, past studies selectively modulated components of the endothelial GABA signaling pathway in vivo. This modulated approach rendered endothelial GABA_{A} receptors dysfunctional and affected GABA release from endothelial cells. The disruption of autocrine and paracrine mechanisms of endothelial cell-mediated GABA signaling had far reaching consequences for brain development, network formation, and subsequently for postnatal behavior. These studies provided novel understanding of how endothelial cell-specific GABA and its receptors signaling shapes neurovascular interactions during embryonic development, and how alterations in this select pathway lead up to psychiatric disease. For instance, embryonic forebrain (telencephalic) angiogenesis was significantly affected and failed to provide physical and chemoattractive guidance for long-distance migration, and final distribution of GABAergic interneurons. It caused a reduction in vascular densities in the embryonic brain, that persisted in the adult brain, with morphological changes in blood vessels indicative of functional changes, accompanied by concurrent GABAergic neuronal cell deficits. This resulted in behavioral dysfunction that was characterized by impaired social recognition, reduced social interactions, communication deficits, increased anxiety and depression and resulted in a new mouse model of psychiatric disorder—the Gabrb3 endothelial cell knockout)(Gabrb3^{ECKO}) mice. These findings are of high significance as they emphasize that the exclusive focus on neuropsychiatric illnesses from a neuronal perspective needs to be broadened to include intrinsic defects within the vasculature that may be the actual trigger for pathophysiological changes.

Currently, psychiatric diseases and/or disorders are treated upon diagnosis or onset of symptoms, which occur after development of physiologic structures, such as blood vessels. However, there is no current treatment for root causes of psychiatric diseases and/or disorders to prevent the onset of any symptoms.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In one embodiment, method for preventing a psychiatric disorder includes providing a therapeutically effective amount of an angiogenesis pathway regulator to an individual.

In a further embodiment, the angiogenesis pathway regulator can cross a utero-placental barrier.

In another embodiment, the angiogenesis pathway regulator is selected from the group consisting of NAD⁺, GABA, VEGF, and FGF.

In a still further embodiment, the angiogenesis pathway regulator is NAD⁺.

In still another embodiment, NAD⁺ is administered at a dose of between 10 mg/kg to 40 mg/kg.

In a yet further embodiment, the administering step is performed orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, or perispinally.

In yet another embodiment, the administering step is performed intraperitoneally.

In a further embodiment again, the individual is pregnant.

In another embodiment again, the offspring of the pregnant individual is susceptible to a psychiatric disorder.

In a further additional embodiment, the psychiatric disorder is selected from the group consisting of autism, epilepsy, schizophrenia, OCD, anxiety, and depression.

In another additional embodiment, the method further includes identifying the individual to be treated.

In a still yet further embodiment, identifying the individual to be treated includes identifying a neurological malformation in the individual.

In still yet another embodiment, the neurological malformation is identified by a CT scan or MRI.

In a still further embodiment again, the individual is identified by measuring NAD⁺ levels in the individual.

In still another embodiment again, a pharmaceutical formulation for the prevention of a psychiatric disorder, includes a therapeutically effective amount of an angiogenesis pathway regulator.

In a still further additional embodiment, the angiogenesis pathway regulator is selected from the group consisting of NAD⁺, GABA, VEGF, and FGF.

In still another additional embodiment, the angiogenesis pathway regulator can cross a utero-placental barrier.

In a yet further embodiment again, the angiogenesis pathway regulator is NAD⁺.

In yet another embodiment again, NAD⁺ is at a dose of between 10 mg to 40 mg.

In a yet further additional embodiment, NAD⁺ is at a dose of 10 mg in 100 μL of saline.

In yet another additional embodiment, the pharmaceutical formulation further includes at least one of the following: a buffer, a stabilizer, a balancer, a flavor, a filler, a disintegrant, a lubricant, a glidant, or a binder.

In a further additional embodiment again, the angiogenesis pathway regulator is formulated for administration orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, or perispinally.

In another additional embodiment again, the angiogenesis pathway regulator is NAD⁺ and is formulated for intraperitoneal administration.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1A illustrates a schema of a defective GABA signaling pathway that can cause psychiatric diseases in accordance with various embodiments of the invention.

FIG. 1B illustrates a schema of an NAD⁺ mediated rescue of a defective GABA pathway in accordance with various embodiments of the invention.

FIG. 1C illustrates a flowchart of a method to treat an individual in accordance with various embodiments of the invention.

FIGS. 1D-1F illustrate exemplary data showing no significant change in behavior in saline and NAD+ treated Gabrb3^{fl/fl} mice.

FIGS. 2A-2L illustrate effects of NAD+ addition to periventricular endothelial cells and neuronal cells isolated from E15 wildtype (CD1) forebrain in accordance with various embodiments of the invention.

FIGS. 3A-3B illustrate summaries of the studies in Gabrb3^{ECKO} mice and schema depicting the paradigm of NAD+ administration in the prenatal period in accordance with various embodiments of the invention.

FIGS. 4A-4O illustrate NAD⁺ mediated rescue of prenatal forebrain angiogenesis and morphological defects in the Gabrb3^{ECKO} telencephalon in accordance with various embodiments of the invention.

FIGS. 5A1-5E illustrate control animals receiving NAD⁺ in the prenatal period also depicted the MGE specific target location in accordance with various embodiments of the invention.

FIGS. 6A-6Y illustrate cellular mechanisms of NAD⁺ rescue in the Gabrb3^{ECKO} telencephalon in accordance with various embodiments of the invention.

FIGS. 7A-7G illustrate Rescue of gene expression profiles in NAD⁺-treated Gabrb3^{ECKO} telencephalon in accordance with various embodiments of the invention.

FIGS. 8A-8C illustrate gene expression profile analysis in accordance with various embodiments of the invention.

FIGS. 9A-9E illustrate NAD⁺ mediated rescue of gene expression in the Gabrb3^{ECKO} telencephalon in accordance with various embodiments of the invention.

FIGS. 10A-10I illustrate rescue of altered gene expresstion by prenatal NAD⁺-treatment in Gabrb3^{ECKO} endothelial cells in accordance with various embodiments of the invention.

FIGS. 11A-11U illustrate molecular mechanisms of NAD⁺ treatment on Gabrb3^{ECKO} endothelial cells in accordance with various embodiments of the invention.

FIGS. 12A-12F illustrate NAD⁺ mediated rescue of calcium signaling related gene expression in Gabrb3^{ECKO} telencephalon in accordance with various embodiments of the invention.

FIGS. 13A-13J illustrate NAD⁺ mediated rescue of calcium influx in Gabrb3^{ECKO} endothelial cells via purinergic signaling in accordance with various embodiments of the invention.

FIGS. 14A-14L illustrate rescue of blood flow and abnormal behaviors in Gabrb3^{ECKO} adult brain after the prenatal NAD⁺ treatment in accordance with various embodiments of the invention.

FIGS. 15A-15L illustrate blood flow changes were observed only in capillaries, and collecting venules, but not in post-capillaries in Gabrb3^{ECKO} cerebral cortex in accordance with various embodiments of the invention.

FIGS. 16A-16J illustrate assays and quantification of behavioral tests performed in saline-treated Gabrb3^{fl/fl}, saline-treated Gabrb3^{ECKO}, and NAD⁺-treated Gabrb3^{ECKO} mice in accordance with various embodiments of the invention.

FIGS. 17A-17G illustrate effects of NAD⁺ and GABA addition to periventricular endothelial cells isolated from E15 wildtype (CD1) forebrains in accordance with various embodiments of the invention.

### DETAILED DESCRIPTION OF THE DISCLOSURE

Turning now to the drawings and data, embodiments of the invention are generally directed to methods of treating and/or preventing psychiatric diseases and/or disorders as well as pharmaceutical formulations configured to treat and/or prevent psychiatric diseases and/or disorders. In many embodiments, the methods and formulations use a regulator of angiogenesis pathways. In several embodiments, the regulators rescue angiogenesis and neurovascular interactions. Specifically, some embodiments rescue telencephalic angiogenesis at prenatal stages to restore downstream neurovascular interactions, normalize brain development, and ameliorate postnatal behavioral symptoms. Many embodiments use at least one of NAD⁺, GABA, VEGF, and FGF as the angiogenesis pathway regulator. Various embodiments treat a psychiatric disease and/or disorder selected from autism, epilepsy, schizophrenia, OCD, anxiety, and depression.

Intrinsic defects within forebrain blood vessels from the earliest developmental time points can be a major cause for the origin of psychiatric diseases. Embryonic forebrain angiogenesis precludes neuronal development and provides valuable guidance cues for neurogenesis and neuronal migration. Many embodiments rescue prenatal forebrain angiogenesis to trigger a rescue of downstream neurovascular interactions. Such rescue provides significant benefits for brain repair during this critical developmental phase.

A natural physiological molecule that can serve to improve cell proliferation and migration would be ideal for in vivo use, during this sensitive gestational time frame. Various embodiments provide the angiogenesis pathway regulator (e.g., NAD⁺) during a window of prenatal development that can serve to rescue angiogenesis and neurovascular interactions in the embryonic telencephalon. NAD⁺ is a co-enzyme found in all living cells and is able to cross the utero-placental barrier. (See e.g., G. J. Burton and A. L. Fowden, The placenta: a multifaceted, transient organ. Philosophical transactions of the Royal Society of London. Series B, Biological sciences 370, 20140066 (Mar. 5, 2015); the disclosure of which is incorporated by reference herein in its entirety.) Additionally, NADPH oxidase in endothelial cells has been reported to generate reactive oxygen species that stimulate angiogenic factors like VEGF, with implications for postnatal angiogenesis in vivo. (See e.g., M. Ushio-Fukai, Redox signaling in angiogenesis: role of NADPH oxidase. Cardiovascular research 71, 226 (Jul. 15, 2006); the disclosure of which is incorporated by reference herein in its entirety.). Additionally, NAD⁺ precursors have been used in the context of aging, Alzheimer's disease, or to relieve postpartum metabolic stress. However, there are no reports of NAD⁺ use and impact in the prenatal developmental period. (See e.g., A. Das et al., Impairment of an Endothelial NAD(+)-H2S Signaling Network Is a Reversible Cause of Vascular Aging. Cell 173, 74 (Mar. 22, 2018); N. Braidy, R. Grant, P. S. Sachdev, Nicotinamide adenine dinucleotide and its related precursors for the treatment of Alzheimer's disease. Current opinion in psychiatry 31, 160 (March, 2018); and P. H. Ear et al., Maternal Nicotinamide Riboside Enhances Postpartum Weight Loss, Juvenile Offspring Development, and Neurogenesis of Adult Offspring. Cell reports 26, 969 (Jan. 22, 2019); the disclosures of which are incorporated herein by reference in their entireties.)

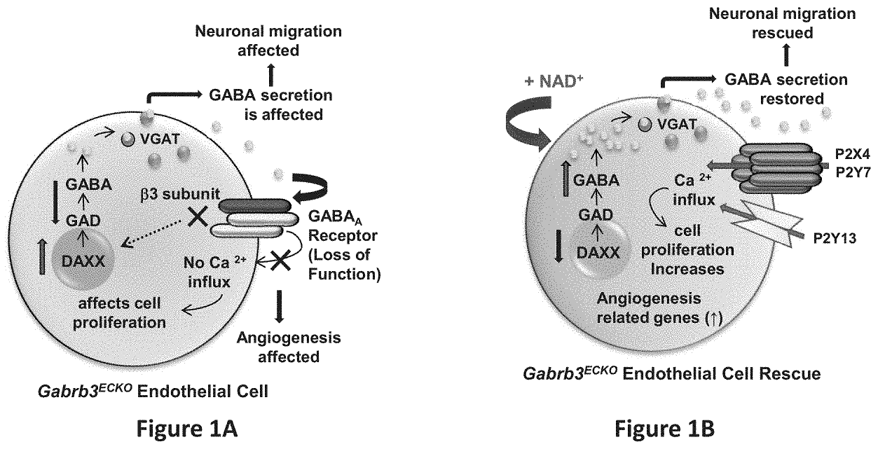

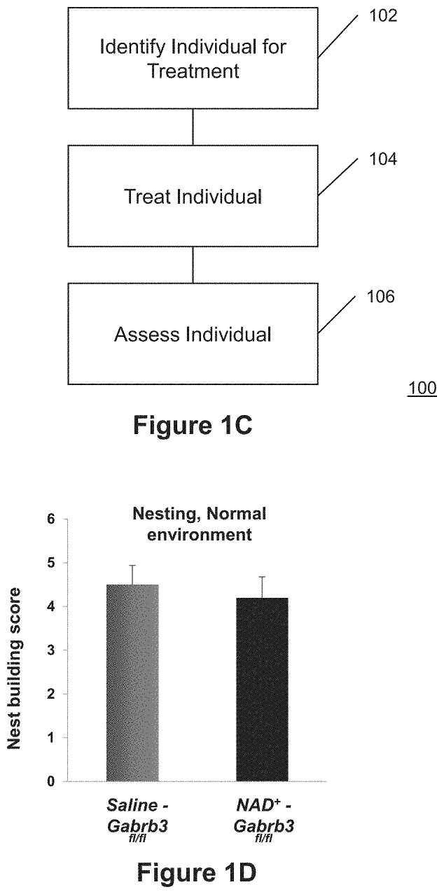

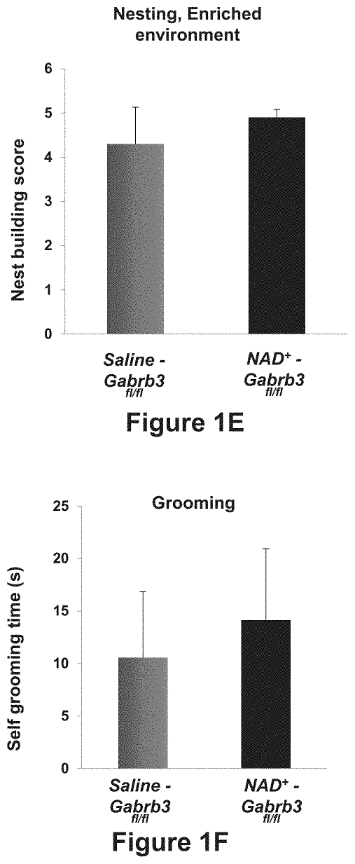

Turning to FIGS. 1A-1B, schematics of neuronal migration are illustrated. Specifically, FIG. 1A depicts defects in the positive feedback GABA signaling pathway in Gabrb3^{ECKO} endothelial cells, in which due to loss of the β3 subunit, GABA_{A} receptors become dysfunctional. As a result, endothelial GABA is unable to activate GABA_{A} receptors and cannot trigger Ca²⁺ influx and endothelial cell proliferation. Gabrb3 also regulates GABA expression via the transcriptional repressor, Daxx. Daxx expression is upregulated in Gabrb3^{ECKO} endothelial cells; therefore, GABA expression is significantly reduced. This affects GABA secretion from Gabrb3^{ECKO} endothelial cells and disturbs paracrine GABA signaling for neuronal migration and autocrine GABA signaling for angiogenesis. FIG. 1B illustrates a NAD⁺ mediated rescue of Gabrb3^{ECKO} endothelial cells in accordance with many embodiments. This NAD⁺ mediated strategy bypasses the GABA_{A} receptor-GABA signaling autocrine pathway and acts via purinergic receptor signaling that triggers Ca²⁺ influx and restores cell proliferation in Gabrb3^{ECKO} endothelial cells. Further, these embodiments cause direct changes to gene expression in Gabrb3^{ECKO} endothelial cells. By downregulating Daxx, it restores GABA expression and secretion in Gabrb3^{ECKO} endothelial cells and thereby restores neuronal migration.

### Compounds

Several embodiments are directed towards compounds and their use as therapeutics to treat and/or prevent psychiatric diseases and/or disorders in an individual. NAD+ in accordance with various embodiments has shown an ability to rescue angiogenesis and morphological malformations or defects, including congenital malformations and defects, in defective telencephalon in vitro as well as to promote GABAergic neuronal development and migration with prenatal treatment. As such, various embodiments utilize NAD+ and/or similar angiogenesis regulators to treat and/or prevent psychiatric diseases and/or disorders. Many of these embodiments use NAD+ as an angiogenesis regulator. However, additional embodiments use GABA, VEGF, and/or FGF (alone or in combination with one or more of the listed compounds).

In many embodiments, NAD+ is utilized as the compound for treatment, as some individuals cannot manufacture or synthesize NAD+ innately from food, vitamins, or other sources. As such, NAD+ precursors may not be successful to treat an individual.

### Pharmaceutical Formulae

Provided herein are various embodiments of pharmaceuticals for use in a treatment or preventative of psychiatric diseases and/or disorders, together with one or more pharmaceutically acceptable carriers thereof and optionally one or more other active ingredients. Proper formulation is dependent upon the route of administration chosen. Any of the well-known techniques, carriers, and excipients may be used as suitable and as understood in the art. Pharmaceutical compositions may be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared utilizing the various method embodiments as described herein.

The term “active ingredient” refers to a compound, which is administered, alone or in combination with one or more pharmaceutically acceptable excipients or carriers, to a subject for treating, preventing, or ameliorating one or more symptoms of a disorder. In various embodiments, active ingredients include one or more of NAD+, GABA, VEGF, and FGF.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al, J. Pharm. Sci. 1977, 66, 1-19.

Numerous coating agents can be used in accordance with various embodiments of the invention. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent. In some embodiments, a disintegrating agent is a super disintegrant agent. In many embodiments, disintegrants are combined with a resin. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Modes of administration, in accordance with multiple embodiments, include, but are not limited to, oral, intravenous, subcutaneous, intramuscular, intrauterine, intraperitoneal, or transmucosal (e.g., sublingual, nasal, vaginal or rectal). The actual amount of drug needed will depend on factors such as the size, age and severity of disease in the afflicted individual. The actual amount of drug needed will also depend on the effective concentration ranges of the various active ingredients. Vehicles of administration, in accordance with various embodiments, include ointments, solutions, gels, creams, suppositories, implants, tablets, or capsules, as appropriate.

In some embodiments, active ingredients are administered in a therapeutically effective amount as part of a course of treatment. As used in this context, to “treat” means to ameliorate and/or prevent at least one symptom of a disease and/or disorder to be treated or to provide a beneficial physiological effect. For example, one such amelioration of a symptom could be vascularization the telencephalon.

A therapeutically effective amount can be an amount sufficient to prevent, reduce, ameliorate, and/or or eliminate the symptoms of at least one psychiatric disease and/or disorder susceptible to such treatment.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the ED50 as determined in cell culture or animal models. Such information can be used to more accurately determine useful doses in humans. Levels in plasma may be measured, for example, by mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day, as determined to be beneficial. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

Preservatives and other additives, like antimicrobial, antioxidant, chelating agents, and inert gases, can also be present. (See generally, Remington: The Science and Practice of Pharmacy, 21st Edition; Lippincott Williams & Wilkins: Philadelphia, Pa., 2005.)

### Methods of Treatment

Several embodiments are directed towards treatments of individuals with compounds or derivatives thereof to treat and/or prevent psychiatric diseases and/or disorders. In some embodiments, compounds or derivatives thereof are administered to an individual having a psychiatric disease and/or disorder, while certain embodiments administer compounds or derivatives thereof to an individual susceptible to having a psychiatric disease and/or disorder. Further embodiments administer compounds or derivatives thereof to a pregnant individual, where the child is susceptible to developing a psychiatric disease and/or disorder or a neurological malformation or defect. In some embodiments, the pregnant individual possesses a psychiatric disease and/or disorder, thus making the child susceptible to developing a psychiatric disease and/or disorder. Certain embodiments treat a child in utero (e.g., a fetus), while some embodiments treat a child shortly after birth, such as a premature child.

Turning to FIG. 1C, an exemplary method 100 for treating an individual for a neurological and/or psychiatric disorder is illustrated. At 102, many embodiments identify an individual to be treated. In some embodiments, the individual is a pregnant mother, where the fetus may possess a structural malformation and/or be at risk for a neurological and/or psychiatric disorder. Being at risk for a neurological and/or psychiatric disorder can include individuals with a family history of such disorders, fetuses showing malformations in a prenatal exam, including CT scan, MRI, and/or any other relevant methodology for identifying neurological structures in utero. Certain embodiments identify an at-risk fetus based on one or more genetic variants in their DNA. Such identification can be accomplished any number of ways, including via an amniocentesis, isolated cell-free fetal DNA (cffDNA), or other methods known in the art. In certain embodiments, the individual is a fetus in utero or a new born infant, including premature infants, for a structural malformation and/or at risk for a neurological malformation and/or psychiatric disorder or disease. Certain embodiments measure NAD+ levels in a person, such as an expectant mother, fetus, or premature baby. Such levels can be measured via a blood draw or other methods that can accurately determine NAD+ levels for the individual.

At 104, certain embodiments treat individuals for a structural malformation and/or at risk for a neurological and/or psychiatric disorder by providing a therapeutically effective amount of an angiogenesis regulator. As used in this context, to “treat” means to ameliorate at least one symptom of the disorder to be treated or to provide a beneficial physiological effect. For example, amelioration of a symptom could be formation or development of typical or natural neurological formations. A therapeutically effective amount can be an amount sufficient to prevent, reduce, ameliorate, or eliminate a neurologic malformation.

Various embodiments utilize NAD⁺ and/or similar angiogenesis regulators to treat and/or prevent neurological and/or psychiatric disorder. Many of these embodiments use NAD⁺ as an angiogenesis regulator. However, additional embodiments use GABA, VEGF, and/or FGF (alone or in combination with one or more of the listed compounds including NAD⁺). Various compounds and formulations for treatment are described elsewhere herein that can be used to treat an in individual in accordance with various embodiments.

Various embodiments provide the compound via one or more suitable methods, such as orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, transdermally, and/or combinations thereof.

In some embodiments, treatment occurs during a period commensurate with neuronal proliferation and migration, which can occur during a time period between approximately 10 weeks and approximately 25 weeks of gestation.

Certain embodiments provide the compound one time, while other compounds provide the compound periodically, such as weekly, monthly, bimonthly, once per trimester, or any other timing to effectively treat an individual. In some embodiments, treatment is performed in the first trimester, while certain embodiments perform treatment in the second trimester, and further embodiments perform treatment in the third trimester. Some embodiments treat throughout the pregnancy at regular intervals (e.g., daily, weekly, every 2 days, every 3 days, every 4 days, every 5 days, every 6 days, etc.). Some embodiments treat preterm or premature children after birth, where brain formation may still be affected by administration of an angiogenesis pathway regulator.

Certain embodiments treat an individual at a dosage of approximately 10 mg/kg—for example, for a 60-70 kg human, a dosage would be approximately 600-700 mg. Some embodiments treat at a dosage higher than 10 mg/kg, such as up to 40 mg/kg. In many embodiments excess NAD+ may be removed from the body without deleterious effects. FIGS. 1D-1F illustrate exemplary data showing no deleterious effects from treating wildtype mice with NAD+. Specifically FIGS. 1D-1E illustrate no significant difference in nesting behavior between saline treated and NAD+ treated Gabrb3^{fl/fl} mice, where 1D shows nesting behavior when provided untorn nestlet, while FIG. 1E illustrates nesting behavior when provided shredded paper. Additionally, FIG. 1F illustrates that NAD+ treated Gabrb3^{fl/fl} mice show no significant change in grooming behavior over saline treated Gabrb3^{fl/fl} mice. As excess NAD+ may be removed without significant effect to a treated individual, some embodiments treat a person prophylactically.

Returning to FIG. 1C, further embodiments assess an individual for a neurological and/or psychiatric disorder at 106. In many embodiments, the assessment is an ongoing process to monitor the individual. In certain embodiments, the individual being assessed is the child or offspring of the person being treated and/or to whom the compound is provided—i.e., a pregnant mother is provided the compound, and the child is assessed.

### EXEMPLARY EMBODIMENTS

Although the following embodiments provide details on certain embodiments of the inventions, it should be understood that these are only exemplary in nature, and are not intended to limit the scope of the invention.

### Methods

The various examples described herein use one or more of the following procedures to generate the described results.

Animals: Timed pregnant CD1 mice were purchased from Charles River laboratories, MA. Colonies of GAD65-GFP mice were maintained in our institutional animal facility. Tie2-cre mice and Gabrb3 floxed (Gabrb3^{fl/fl}) mice were obtained from Jackson Labs. The Tie2-cre transgene is known for uniform expression of cre-recombinase in endothelial cells during embryogenesis and adulthood. To selectively delete Gabrb3 in endothelial cells, Tie2-cre transgenic mice (males) were crossed to Gabrb3^{fl/fl} mice (females) to generate Tie2-cre; Gabrb3^{fl/+} mice (males). These were further crossed with Gabrb3^{fl/fl} mice (females) to obtain the Gabrb3 conditional knock-outs (Tie2-cre; Gabrb3^{fl/fl} mice). The day of plug discovery was designated embryonic day 0 (E0). Animal experiments were in full compliance with the NIH Guide for Care and Use of Laboratory Animals and were approved by the HMRI and McLean Institutional Animal Care Committees.

Histology, immunohistochemistry, and microscopic analysis: Paraffin immunohistochemistry (IHC) was performed on embryonic brains only while frozen section IHC was used on both embryonic and adult brains. Briefly, for paraffin IHC-E18 brains were fixed in zinc fixative (BD Biosciences PharMingen) for 24 h and processed for paraffin histology. Histological stainings with hematoxylin (Vector Laboratories) and eosin (Sigma) were performed on 8 μm coronal sections. Lectin histochemistry (with biotinylated isolectin B4, 1:50, Sigma) as well as IHC was performed on 20 μm sections. Primary antibodies used for IHC were as follows: anti-PHH3 (1:200, Millipore) and anti-NKX2.1, (1:50; Sigma) followed by secondary detection with AlexaFluor conjugates (Invitrogen). DAPI (Vector Laboratories) was used to label nuclei. For frozen section IHC, E18 and P90 brains were removed, fixed in 4% PFA for 24 hours, cryo-protected in sucrose gradient, embedded into OCT medium for frozen blocks; sectioned at 40 μm on a cryostat and immunostained with anti-GABA (1:80, Sigma) and anti -PROX1 (1:80, Millipore) antibodies. Twenty sections from each brain were used for IHC and histology experiments. Uniform penetration of antibodies or stains throughout the section was ascertained and quality of the staining in each digital section was examined. Only those sections which showed uniform labeling were included in further analysis. All low and high-magnification images were obtained from an FSX100 microscope (Olympus).

Morphometry: A stereological point grid was superimposed on digital images of biotinylated isolectin-B4+ vessels using ImageJ software. The ratio between points falling on blood vessels and on brain tissue was calculated for each section, and average values were obtained.

Cell counting: Profiles of GABA+ immunoreactive cells were counted in the prefrontal cortex (at bregma levels 1.5, 0.5 and −1.5) using stereotaxic coordinates from the atlas: The Mouse Brain in Stereotaxic Coordinates, by Paxinos and Franklin. For each area, cells in the strip of cortex from the pial surface to the white-gray matter interface were counted using ImageJ software and plotted.

Primary culture of endothelial cells and endothelial cell staining: Embryonic brains were dissected under a stereo-microscope, after the NAD⁺ treatment paradigm and the telencephalon was removed. Pial membranes were peeled off. Telencephalon without pial membranes from the periventricular region was pooled. Purity of endothelial cell cultures was established with endothelial cell markers and determined to be one hundred percent. Isolation and culture of endothelial cells were performed according to published methodology. Periventricular endothelial cells were prepared from CD1 (wild type), saline-treated Gabrb3^{fl/fl}, saline-treated Gabrb3^{ECKO}, and NAD⁺-treated Gabrb3^{ECKO} embryos. Endothelial cells were labeled with anti-biotinylated isolectinB4 (1:100, Sigma), anti-GABA (1:400, Sigma), anti-DAXX (1:100, Santa Cruz Biotechnology) and anti-P2X4 (1:100, Abclonal) followed by secondary detection with AlexaFluor conjugates (Invitrogen). DAPI (Invitrogen) was used to label nuclei. Images were taken on an FSX100 microscope (Olympus). 1 million cells were examined for each immunohistochemistry condition.

Endothelial cell proliferation and long-distance cell migration assay: To test for cell proliferation, CD1 periventricular endothelial cells (1 million cells per experiment) were incubated in the presence of the mitotic marker 5-bromo-2′-deoxyuridine (0.05% BrdU) with or without muscimol, for 1 hour to examine the impact on proliferation of these cells and processed for BrdU immunohistochemistry.

In preparation for long-distance cell migration assays, square culture inserts (ibidi GmbH) were placed at one end of a 35 mm dish. Cultures of CD1 derived endothelial cells, purified from the periventricular plexus, were plated in the insert and allowed to migrate for 24 hours in endothelial cell culture medium. Endothelial cells were labeled with cell trace marker (CellLight Plasma Membrane-RFP, BacMam 2.0, Invitrogen) to visualize endothelial cell morphologies during subsequent imaging. The migration of endothelial cells from one end of the dish to the other spanning a distance of 3.5 cm was imaged and quantified.

Isolation and primary culture of neuronal cells: Primary culture of embryonic GABAergic neurons from CD1 telencephalon or after the saline or NAD+ treatment paradigms were performed using established methods. Briefly, embryonic brains were extracted under a stereo microscope and placed into cold PBS. After removal of the pial membrane, the telencephalon was dissected from each embryonic brain. The telencephalon was minced into 1-2 mm slices in cold PBS. Minced telencephalon was treated with 0.1× trypsin/EDTA at 37° C. for 5 min. Trypsin treatment was stopped by adding FBS-DMEM media and DNase I consecutively. Dissociated cells were filtered with a 40 μm cell strainer and finally filtered cells were cultured in poly-D-lysine coated culture dishes with Neurobasal medium (Life technologies) with 1× B-27 (Life technologies) and 1× Glutamax (Life technologies).

Neuronal migration assay: In preparation for cell migration assays, one well square culture inserts (ibidi GmbH) were placed in the center of a poly-Ornithine/Laminin coated 35 mm dish, to make a small rectangular patch that is labeled outside the dish. Embryonic neurons prepared from the saline and NAD+ treated groups were seeded in the inserts in Neurobasal medium, supplemented with 1× B-27 and 1× Glutamax. After the cells had attached, the inserts were removed to initiate cell migration in all 4 quadrants of the dish. After 24 hours, cells were fixed and labeled with anti-β-Tubulin antibody (1:2000, Biolegend). Neuronal migration was assessed by measuring the distance between the final positions of cells from the border of the rectangular patch was outlined on the first day, using ImageJ software.

Calcium imaging: For Ca²⁺ assays, periventricular endothelial cells (1 million cells per assay) were incubated with the Ca²⁺ indicator dye FluoForte AM according to manufacturer's instructions (Enzo Life Sciences), loaded into the chamber of an FSX 100 microscope and imaged continuously before and after αβ-meATP, Bz-ATP and 2Me-SADP application (100 μM). Fluorescence micrographs were digitalized and results were expressed as change in fluorescence over baseline fluorescence.

Gene expression profile analysis: RNA samples were prepared from saline-treated Gabrb3^{fl/fl}, saline-treated Gabrb3^{ECKO} and NAD⁺-treated Gabrb3^{ECKO} groups from three different brain pools. Total RNA from each of the samples was extracted by using the PicoPure RNA Isolation kit (Arcturus) following the supplier's protocol. Microarray was performed with Mouse Gene 2.0 ST Array (Affymetrix) at the Boston University Microarray & Sequencing Resource, Boston, MA. Mouse Gene 2.0 ST CEL files were normalized to produce gene-level expression values using the implementation of the Robust Multiarray Average (RMA) in the affy package (version 1.36.1) included in the Bioconductor software suite (version 2.11). Principal component analysis (PCA) was analyzed and visualized using Transcriptome Analysis Console 4.0 (Affymetrix). Heatmap visualization and analysis were performed using Morpheus (Broad Institute, Boston, Mass., USA; software.broadinstitute.org/GENE-E/), and ranked by t-test statistics. Violin plot visualization was generated with Z-score using GraphPad Prism v8.0 (GraphPad Software, La Jolla Calif. USA). The gene ontology for gene enrichment study was performed in three GO TERM annotation categories by using the Database for Annotation, Visualization and Integrated Discovery (DAVID) v6.8. Mouse Genome Informatics (MGI) (GO TERM structure categories: cerebral cortex interneuron migration; GABAergic neuron differentiation; interneuron development; MGE derived cells).

Quantitative real-time PCR: RT was performed by using iScript Reverse Transcription Supermix kit (Bio-Rad). PCR reactions were run on a CFX96 Touch Real Time PCR (Bio-Rad) with SsoAdvanced™ Universal SYBR® Green Supermix (Bio-Rad). Primers for qPCR (Daxx, P2x4, P2x7, P2y13, Tek, vwf, F2r, Sirt2, Nos1, Nrp1, Pax6, Gad1, Gad2, Grin2a, Trpm2, Trpc4 and Trpv6) were obtained from Thermo Fisher Scientific. The housekeeping gene Gapdh was used as a reference. The relative gene expression and subsequent fold changes among different samples were determined according to published methodology.

ELISA: Periventricular endothelial cells were prepared after the NAD⁺ treatment paradigm, or after the treatment with purinergic receptor agonists/antagonists, and seeded in 12 well culture plates at 0.1×106 cells/well. Supernatants from endothelial cell cultures were collected after 96 hours and stored at −80° C. for ELISA. GABA concentrations were quantitatively determined by competitive ELISA according to the manufacturers' protocol (GABA Research ELISA kits, Labor Diagnostica Nord, Germany), and absorbance was measured using a multiplate microplate fluorescence reader (Molecular Devices, CA) at 450 nm.

In vivo imaging of brain microvasculature by multiphoton laser-scanning microscopy: In vivo imaging of the brain vasculature in cranial window bearing mice was performed as described previously. Briefly, a cranial window was implanted by removing circular area of skull and dura. Then, the window was sealed with a 7 mm cover glass glued to the bone. For the measurement of red blood cell (RBC) velocity and blood vessel diameter, we used multiphoton laser-scanning microscopy (MPLSM). To avoid potential tissue/vessel alteration caused by the window implantation procedure, we performed imaging at least 10 days after cranial window implantation. For imaging, mice were anesthetized with ketamine/xylazine, then tetramethylrhodamine (TAMRA)-dextran (MW 500,000) was administrated through retro-orbital injection. Using TAMRA-dextran contrast enhanced angiography, region of interest is first identified. Since the intravenously injected dye labels only the blood plasma, RBCs appear as dark patches moving within the vessel lumen. Centerline RBC velocity was measured by repetitively scanning a line along the central axis of a single blood vessel (x-t) and enabling the tracking of the motion of these dark patches. The space-time image produced by the line-scan contained diagonal dark streaks formed by moving RBCs, with a slope that was inversely proportional to the centerline RBC velocity. This space-time image was then computationally processed using Matlab and Python to extract the gradient of each streak, corresponding to the RBC velocity. Briefly, diagonal filters of varying gradients were tested for each streak until the gradient that fit best was found. This was conducted for each streak in the space-time image, and the mean gradient was taken. For the vessel diameter, an edge filter was applied to determine the blood vessel boundaries, and the blood vessel diameter extracted at the indicated region of interest. The blood flow rate was determined through the following formula:

$$\dot{Q} = π {r}^{2} \dot{v}$$

Where [symbol] is the final flow rate, r is the vessel radius, and [symbol] is the blood flow velocity.

Behavioral Experiments: Mice were housed in our animal facility with a 12-hour light cycle with ad libitum access to food and water. Offspring stayed with their mothers until weaning at PND 21 after which males and females were separated. Before all behavioral testing, mice were acclimated to the testing room for 1 hour. Behavioral assays were performed according to established protocols referenced here: self-grooming, light-dark box, tail suspension test, Y-maze, open field locomotion activity, three chamber social interaction test and nest building with nestlets and with shredded paper. Both males and females were used for all behavioral assays. Experimenters scoring behaviors were blinded to the genotypes and treatment. Sample sizes for each assay are noted in figure legends.

Statistical analysis: For each experiment, samples were collected from either 1 or 2 embryos of the same genotype or postnatal mice from a given litter. Five to 10 litters of mice for each prenatal experiment and 3 to 10 litters of mice for each postnatal experiment were used. Thus, data from 8 to 10 individuals per prenatal and postnatal condition were used. For behavioral experiments, 8 to 16 litters of mice were used. Statistical significance of differences between groups was analyzed by either one-way ANOVA or two-tailed Student's t test (Prism; GraphPad software). Significance was reported at p<0.05.

### Example 1: Prenatal NAD⁺ Treatment Rescues Angiogenesis and Morphological Defects in the Gabrb3^{ECKO} Telencephalon

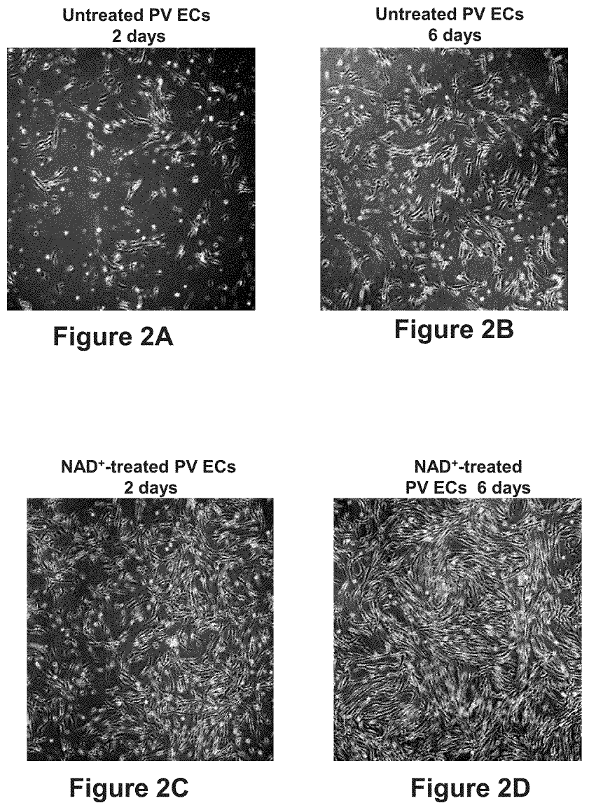

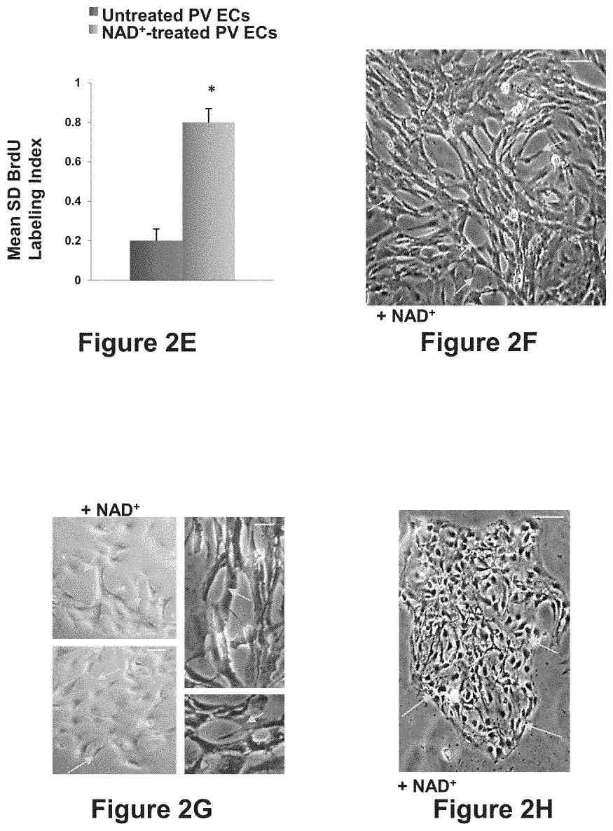

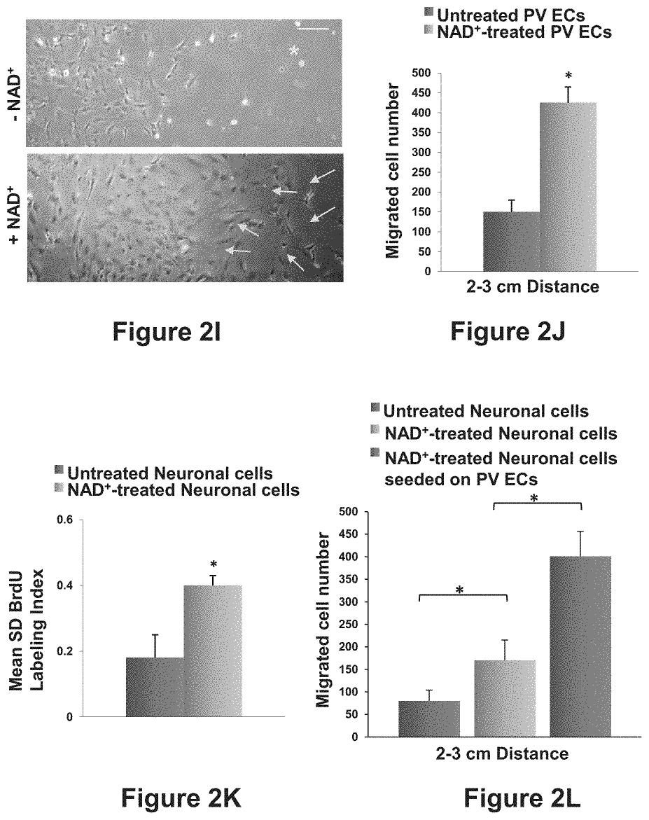

In one experiment, periventricular endothelial cells were isolated from E15 wildtype (CD1) forebrain and tested the effect of NAD⁺ addition in vitro. Exogenous addition of 100 μM NAD⁺ was able to initiate significant endothelial cell proliferation within 2 days of culture and a 35 mm culture dish was confluent within 4-6 days (FIGS. 2A-2D). This was an interesting observation, since untreated periventricular endothelial cells take approximately 12 days to reach confluency in 35mm dishes, in our well-established culture conditions. Endothelial cell bromodeoxyuridine (BrdU) labeling index was significantly increased, indicating that NAD⁺ was able to robustly trigger endothelial cell proliferation (FIG. 2E). Vital elements of angiogenesis include effective endothelial cell proliferation, migration, sprouting, alignment, tube formation and branching. Interestingly, NAD⁺-treated periventricular endothelial cells started showing tube formation properties under normal culture conditions, which was highly unusual in the absence of a substrate such as Matrigel (FIGS. 2F-2H). The NAD⁺-treated endothelial cells demonstrated tubular network formation in the absence of a three-dimensional milieu, with budding, branching and lumen formation (FIGS. 2F-2G). In some areas of the dish, the NAD⁺-treated endothelial cells seemed to align and fold into tubular shapes (FIG. 2H), aspects that we have not seen in wild type or untreated periventricular endothelial cells in normal culture conditions. NAD⁺ treated endothelial cells also showed robust long-distance migration, from one end of the dish to the other, in terms of cell number, when compared to untreated cells (FIGS. 2I-2J). Collectively, these results confirmed the high angiogenic potential of NAD⁺ addition on periventricular endothelial cells. The effects of NAD⁺ addition on neuronal cells isolated from the E15 CD1 forebrain were also tested and found an increase in cell proliferation (FIG. 2K). NAD⁺ addition similarly increased long-distance migration of neuronal cells, but the effect was more robust when neurons were seeded on periventricular endothelial cells (FIG. 2L). Though the endothelial cell response to NAD⁺ (FIGS. 2E & 2J) was more pronounced than neuronal cells alone (FIGS. 2K-2L), these results provided convincing evidence that the NAD⁺ addition was having positive effects on both cell types, and formed the rationale for exploring the use of NAD⁺ in vivo.

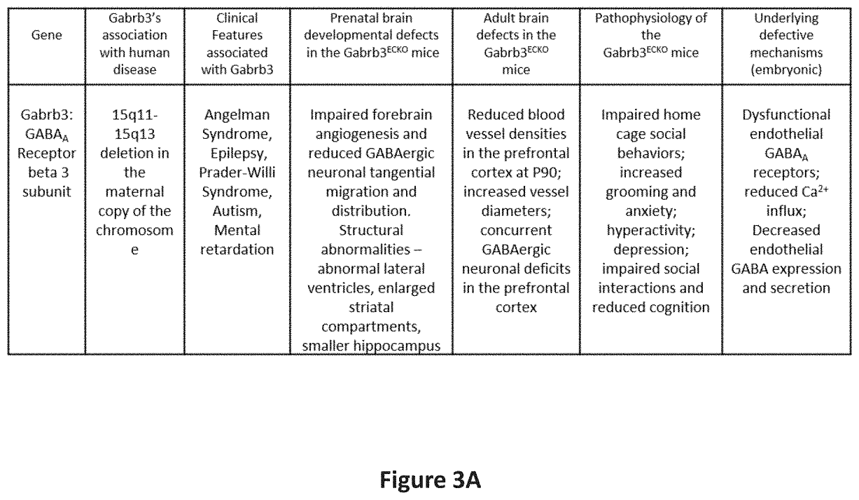

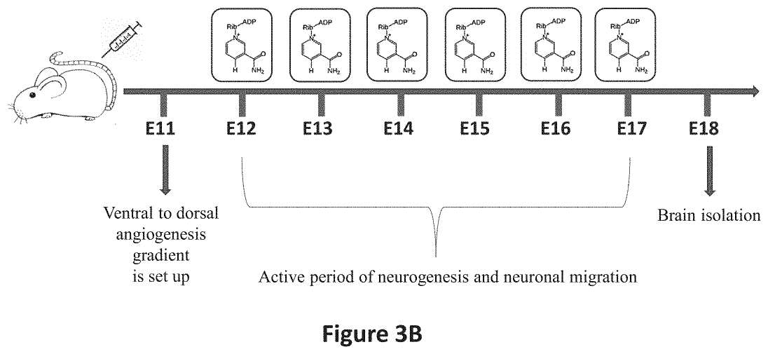

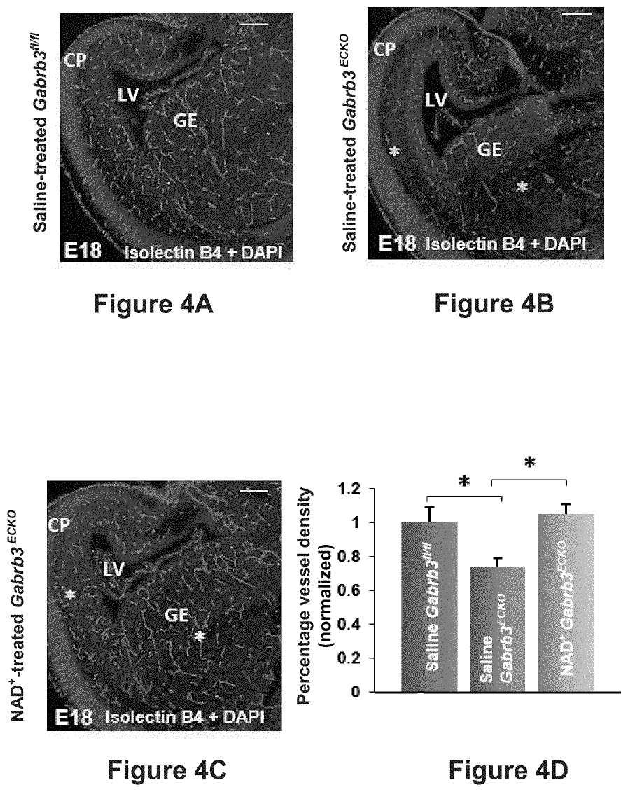

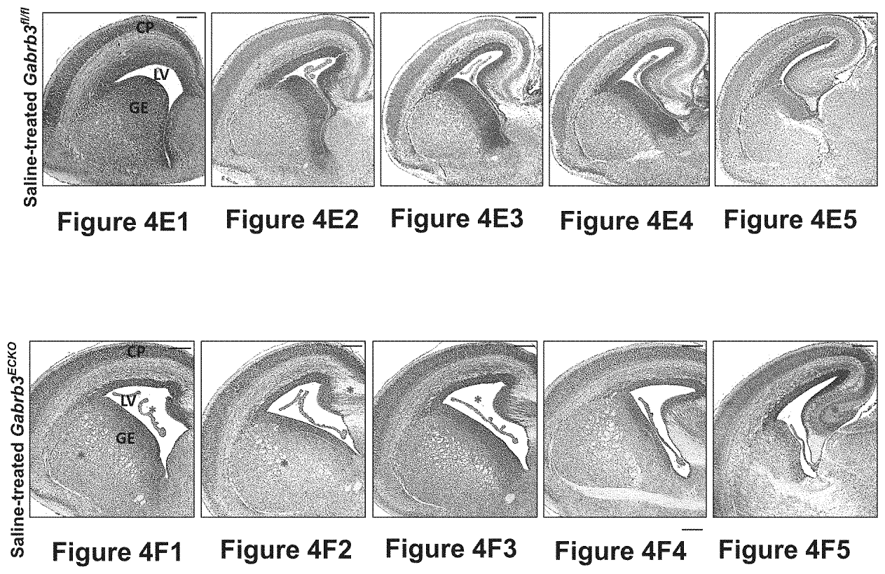

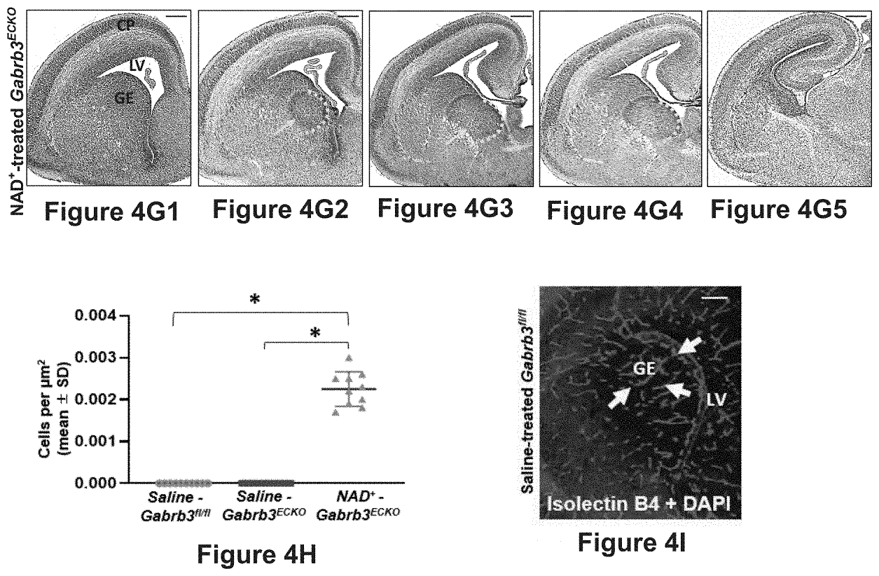

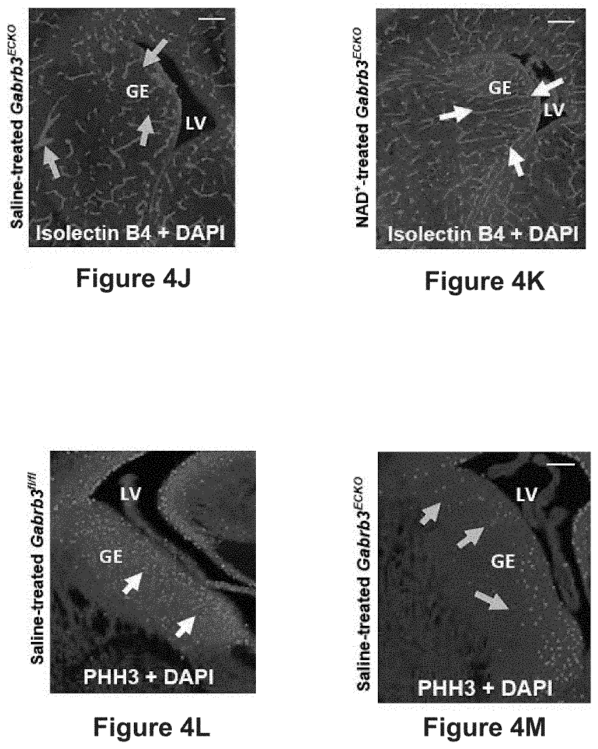

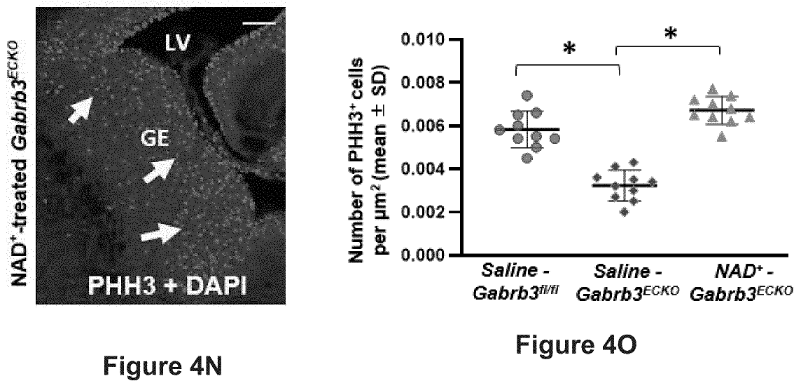

In the Gabrb3^{ECKO} telencephalon, labeling with multiple markers of vessel components, isolectin B4 and CD31/PECAM-1 have revealed reductions in vessel density and pattern formation from embryonic day 13 (E13) onward to E18. This vascular deficit in the embryonic telencephalon persisted in the adult cerebral cortex with larger vessel diameters likely correlating with increased perfusion and indicative of functional changes in blood flow in Gabrb3^{ECKO} vessels. A summary of the embryonic and postnatal phenotype highlights (FIG. 3A) the importance of the endothelial GABA pathway for telencephalic angiogenesis, and for maintaining neuro-vascular interactions. Therefore, given its pro-angiogenic properties (FIGS. 2A-2J), NAD⁺ treatment during the prenatal period was tested for its ability to improve angiogenesis in the Gabrb3^{ECKO} telencephalon. The periventricular ventral-dorsal angiogenesis gradient is established by E11, after which neuronal cells originating from ventricular zones navigate along radial and tangential courses, to adopt final laminar positions and integrate into specific brain circuits. Therefore, the approach was to target only the critical window of mouse prenatal brain development: E12 to E17. In the Gabrb3^{ECKO} telencephalon, the periventricular vessel gradient is formed normally at E11, but reductions in vascular density and abnormal vascular profiles are observed from E13 onward. Hence, NAD⁺ treatment (10 mg in 100 μl of saline) or a placebo solution (100 μl of saline) was given intraperitoneally, daily to pregnant dams from E12 to E17 (FIG. 3B). Three groups were compared throughout the study: Group 1: Saline-treated Gabrb3^{fl/fl} mice or controls; Group 2: Saline-treated Gabrb3^{ECKO} mice; and Group 3: NAD⁺-treated Gabrb3^{ECKO} mice. At E18, brains were isolated and vascular densities were assessed by labeling blood vessels with biotinylated isolectin B4 in paraffin sections. Interestingly, a significant improvement in periventricular vessel densities was observed in NAD⁺-treated Gabrb3^{ECKO} telencephalon versus saline-treated Gabrb3^{ECKO} telencephalon and was comparable to saline-treated Gabrb3^{fl/fl} telencephalon (FIGS. 4A-4D). These results implicated that the prenatal NAD⁺ treatment was successful in rescuing angiogenesis in the Gabrb3^{ECKO} telencephalon.

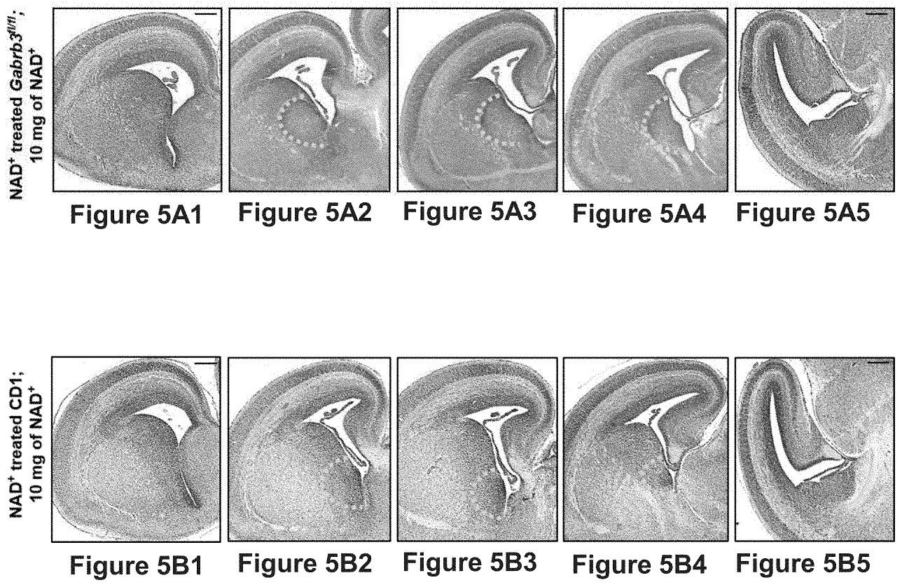

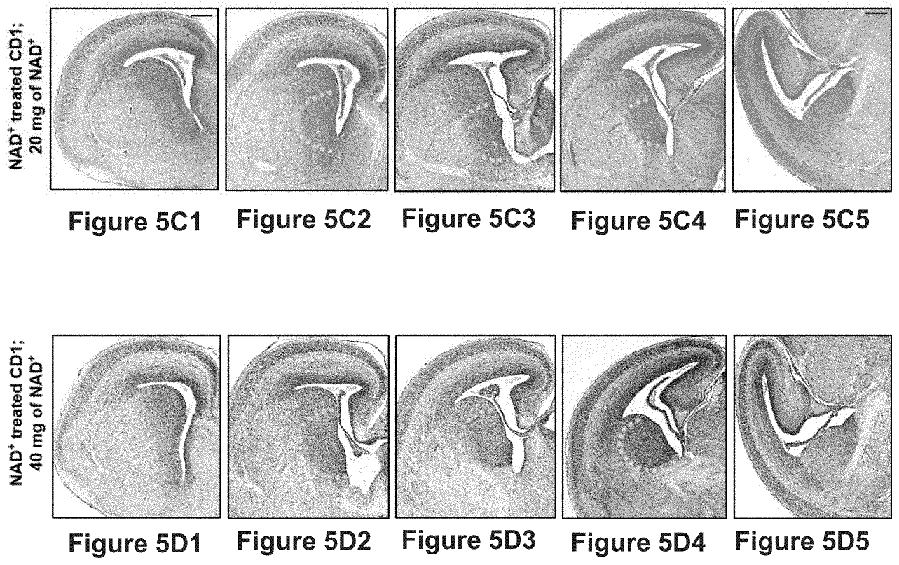

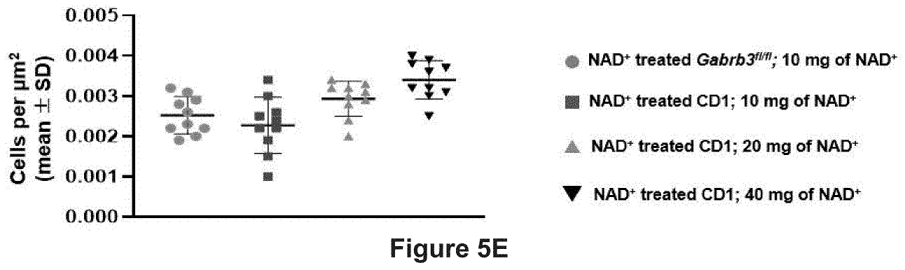

Furthermore, another finding was made when the morphology of NAD⁺ treated Gabrb3^{ECKO} telencephalon was examined at E18 (FIGS. 4E1-4G5). In histological stainings, we have previously reported morphological defects in the Gabrb3^{ECKO} medial telencephalon at E18 along with marked ventricular abnormalities, reduced hippocampus and enlarged striatal compartments and these perturbations in anatomical landmarks were consistently observed in saline-treated Gabrb3^{ECKO} telencephalon at all rostro-caudal levels (FIGS. 4F1-4F5). Of importance, NAD⁺ treatment during E12 to E17 significantly improved the overall morphology of the Gabrb3^{ECKO} telencephalon (FIGS. 4G1-4G5), restored anatomical landmarks and ventricular size, and was similar to saline controls (FIGS. 4E1-4E5). Interestingly, in the H&E staining, a cluster of cells were observed in the medial ganglionic eminence (MGE) of NAD⁺-treated Gabrb3^{ECKO} mice that were discernible specifically in the middle sections along the rostro-caudal axis. This seemed to indicate that the prenatal NAD⁺ treatment had a selective target in the MGE, that was quantified (FIG. 4H). Such a distinct type of cellular arrangement was not seen in other regions of the forebrain or in the midbrain and hindbrain of NAD⁺-treated Gabrb3^{ECKO} mice. NAD⁺-treated Gabrb3^{fl/fl} littermates were evaluated, which possessed the same distinct cellular arrangement in the MGE indicating that the treatment targeted the same region in all genotypes (FIGS. 5A1-5A5). An increasing dose regimen of NAD⁺ treatment (10 mg, 20 mg and 40 mg per mouse; i.p.) was performed in wildtype CD1 mice to test if this observation was consistent in a different strain of mice and observed a similar cell cluster in the MGE (FIGS. 5B1-5D5), indicating that the target was region-specific. The lowest effective dose (10 mg per mouse) was therefore used in all of the experiments in this study.

Encircling the lateral ventricle is a rich tube-like plexus of vessels which serves as a niche for neuronal proliferation and migration. This unique curved profile of vessels can be observed even in 20 μm thick sections from saline controls (FIG. 4I), but was disrupted in the ganglionic eminence of the saline-treated Gabrb3^{ECKO} telencephalon (FIG. 4J). In NAD⁺-treated Gabrb3^{ECKO} telencephalon, these uniform vascular patterns were restored (FIG. 4K); reinforcing its positive effects on in vivo angiogenesis. Cell proliferation was analyzed in the ventral telencephalon by examining interkinetic nuclear migration with phosphohistone 3 (PHH3), a specific marker for cells undergoing mitosis (FIGS. 4L-4O). Abnormal PHH3⁺ profiles were observed in the saline-treated Gabrb3^{ECKO} telencephalon (FIG. 4M) when compared to saline-treated controls (FIG. 4L). NAD⁺ treatment significantly increased the number of PHH3⁺ cells at the VZ/SVZ surface of Gabrb3^{ECKO} ventral telencephalon, indicative of increased endothelial and neuronal cell proliferation (FIG. 4N).

### Example 2: Prenatal NAD⁺ Treatment Promotes GABAergic Neuronal Development and Migration

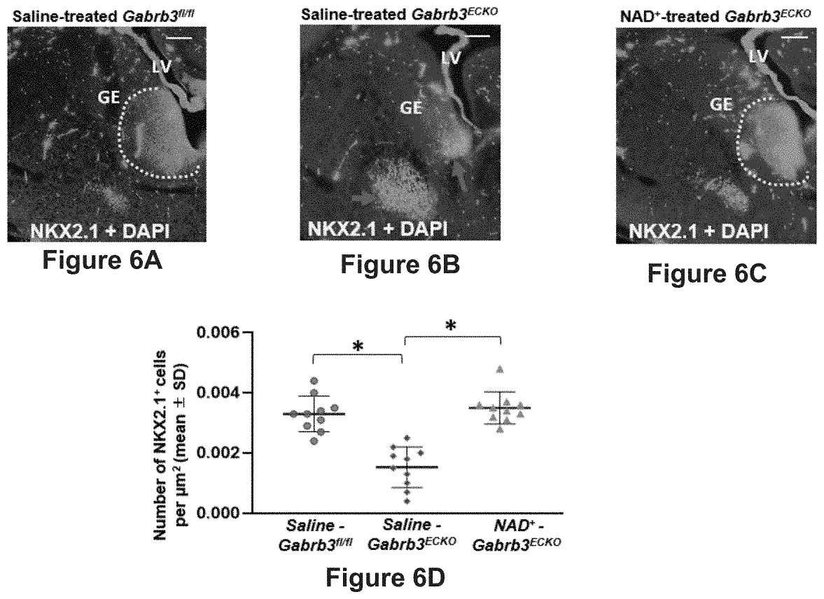

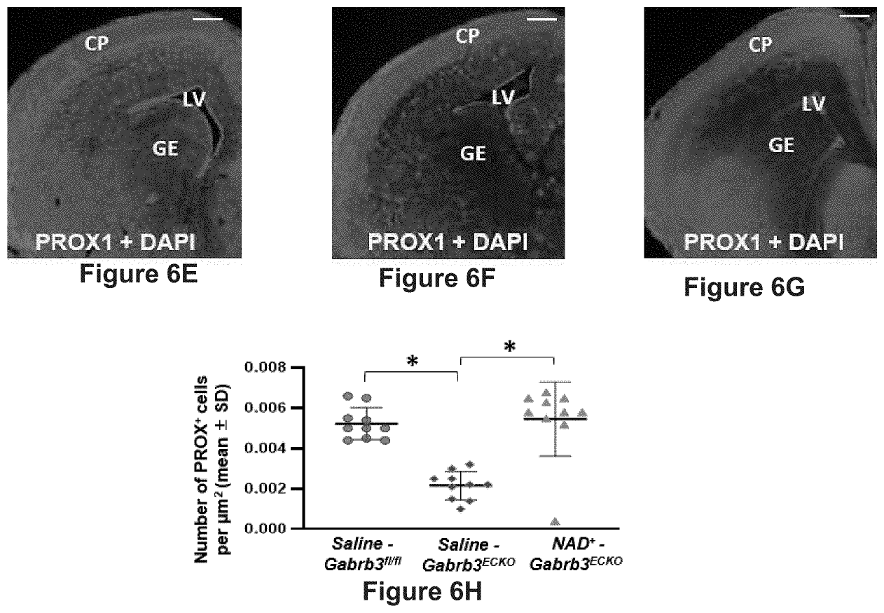

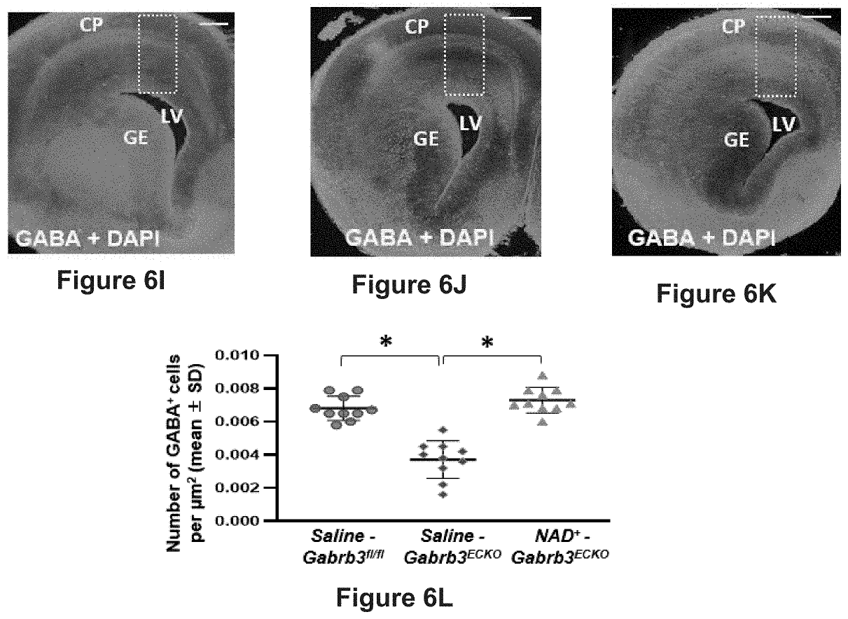

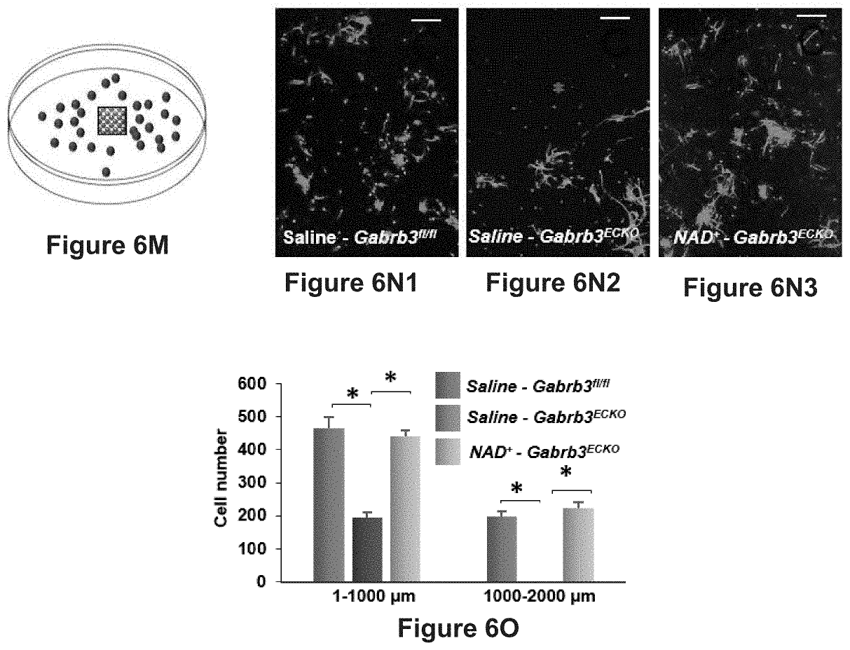

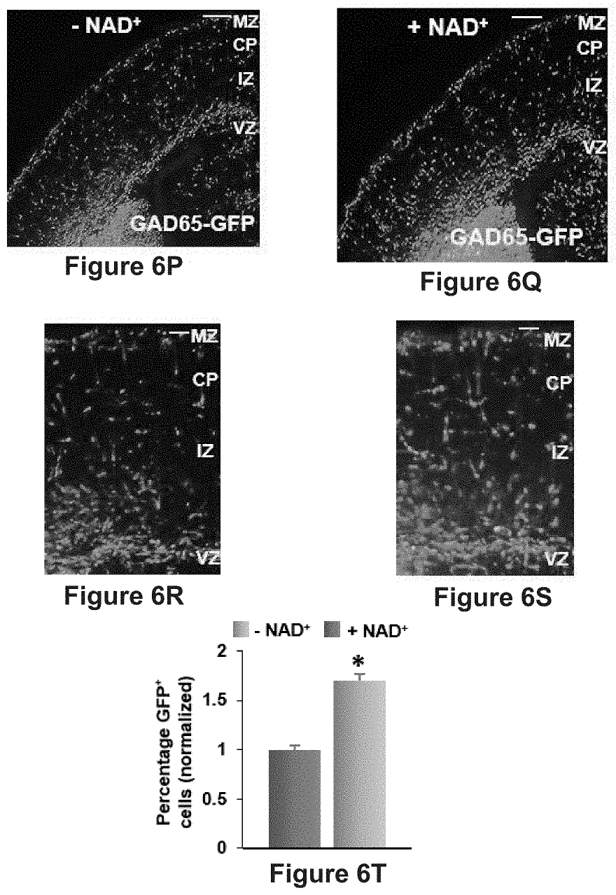

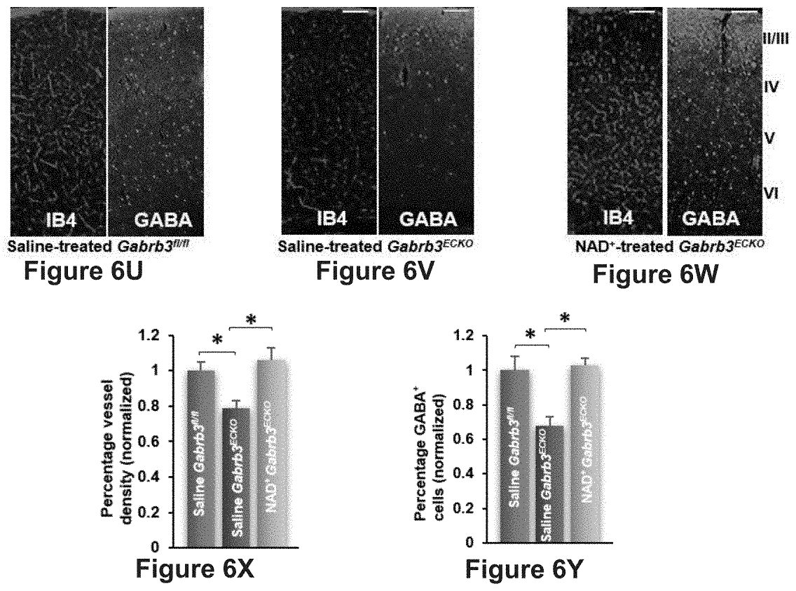

In another experiment, the significance of the prenatal NAD⁺ treatment on cellular mechanisms in the E18 Gabrb3^{ECKO} telencephalon was evaluated with comparisons to the saline-treated groups. Expression of the homeodomain protein NKX2.1 was tested for, which is specifically expressed by MGE progenitors and is selective to cells of the ventral telencephalon (preoptic area, MGE, globus pallidus, septum and amygdala). Nkx2.1 mutants lack an MGE and have interneuron loss in the cerebral cortex. Nkx2.1 also acts as a cell fate determinant in regulating the differential migration of cortical and striatal GABAergic interneurons. We observed a significant reduction of NKX2.1 expression in the MGE of the saline-treated Gabrb3^{ECKO} telencephalon (FIGS. 6B & 6D) when compared to the saline-treated Gabrb3^{fl/fl} telencephalon (FIGS. 6A & 6D). Abnormally higher expression of NKX2.1 was observed in the globus pallidus which may be indicative of arrested or stalled migration, and GABAergic neurons are therefore more likely to remain in the basal forebrain (FIG. 6B). This abnormal NKX2.1 profile in the Gabrb3ECKO ventral telencephalon was rescued by the prenatal NAD+ treatment. NKX2.1 expression was significantly enriched in the MGE of the NAD⁺-treated Gabrb3^{ECKO} telencephalon (FIGS. 6C & 6D). Expression of PROX1 was tested for, which is a marker that defines LGE/CGE/POA-derived cortical interneurons and is expressed in dividing precursors, immature migrating interneurons as well as mature fully integrated cells. While the saline-treated Gabrb3^{fl/fl} telencephalon showed a normal PROX1 profile (FIGS. 6E & 6H), a decrease in PROX1 immunoreactivity was observed in saline-treated Gabrb3^{ECKO} telencephalon (FIGS. 6F & 6H). PROX1 expression was significantly increased in the NAD⁺-treated Gabrb3^{ECKO} telencephalon (FIGS. 6G & 6H). The profile of GABAergic neurons, examined with GABA immunoreactivity, was reduced in saline-treated Gabrb3^{ECKO} telencephalon (FIGS. 6J & L), but was restored in NAD⁺-treated Gabrb3^{ECKO} telencephalon (FIGS. 6K & L) similar to the control (FIGS. 6I & 6L). These results provided novel evidence that in addition to the improvement in vascular profiles and cell proliferation in the ganglionic eminence, the distinct cellular architecture observed in the MGE of the NAD⁺-treated Gabrb3^{ECKO} telencephalon was pro-GABAergic and consisted of NKX2.1⁺ and PROX1⁺ cells with significance for GABAergic neuronal development.

To test if the prenatal NAD⁺ treatment was able to influence GABAergic neuronal migration, an in vitro neuronal migration assay was performed (FIG. 6M), after the in vivo NAD⁺ treatment paradigm (E12-E17). GABAergic neurons were isolated from the three groups at E18 by using established methodology and seeded in culture inserts in the center of 35 mm laminin coated culture dishes (FIGS. 6M & 6N1-6N3). β-Tubulin⁺ neurons from saline controls and NAD⁺-treated Gabrb3^{ECKO} groups migrated robustly (FIGS. 6N1, 6N3, & 6O), unlike neurons from the saline-treated Gabrb3^{ECKO} group (FIGS. 6N2 & 6O), both in terms of cell number and distance. These results implicated that the prenatal NAD⁺ treatment was able to improve the intrinsic capacity of neuronal migration in Gabrb3^{ECKO} neurons. This aspect was further explored by using the E12-E17 NAD⁺ injection paradigm in GAD65-GFP knock in mice (FIGS. 6P-6T). Images of GFP⁺ cells that had entered the dorsal telencephalon at E18 were collected and analyzed. A visual inspection of the sections revealed an increase in the distribution of the GFP⁺ cells in the NAD⁺-treated group (FIGS. 6Q & 6S) versus the NAD⁺-untreated group (FIGS. 6P & 6R). Numerous GFP⁺ cells were found extending throughout the lateral to medial expanse of the NAD⁺ treated dorsal telencephalon (FIGS. 6R-6S). Quantification also revealed a significant increase in the percentage of GFP⁺ cells in the NAD⁺-treated telencephalon (FIG. 6T). This data suggested that prenatal administration of NAD⁺ can regulate neuronal migration and will be beneficial in disease models in which deficits in GABAergic neuronal development and migration are reported.

Next this exemplary embodiment tested whether the prenatal rescue of blood vessel densities, angiogenesis and GABAergic neuronal profiles by NAD⁺ will persist in the adult Gabrb3^{ECKO} cerebral cortex (FIGS. 6U-6Y). The vascular and GABA cell deficit observed in the saline-treated Gabrb3^{ECKO} embryonic brain (FIGS. 4B, 4D, 6J & 6L) was also recapitulated in the saline-treated Gabrb3^{ECKO} prefrontal cortex (P90) as expected (FIGS. 6V, 6X, & 6Y). However, a concurrent increase in blood vessel densities and GABAergic interneurons was observed in the NAD⁺-treated Gabrb3^{ECKO} cerebral cortex (FIGS. 6W, 6X, & 6Y) and was comparable to saline-treated controls (FIGS. 6U, 6X, & 6Y) indicative of long-lasting rescue initiated by the prenatal NAD⁺ treatment.

Collectively, these results implicated a rescue of endothelial and neuronal cellular mechanisms by prenatal NAD⁺ treatment in the Gabrb3^{ECKO} forebrain and raised new questions about the molecular mechanisms of NAD⁺ action and rescue.

### Example 3: NAD⁺ Mediated Rescue of Gene Expression Profiles in Gabrb3^{ECKO} Telencephalon

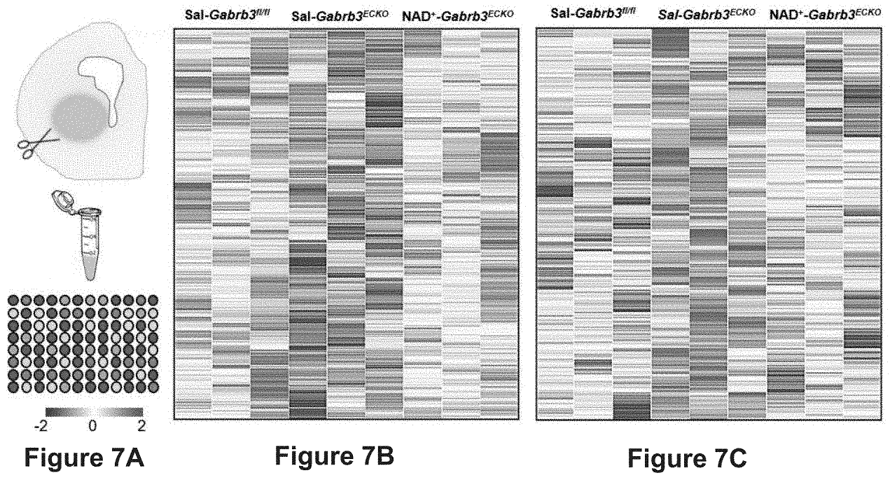

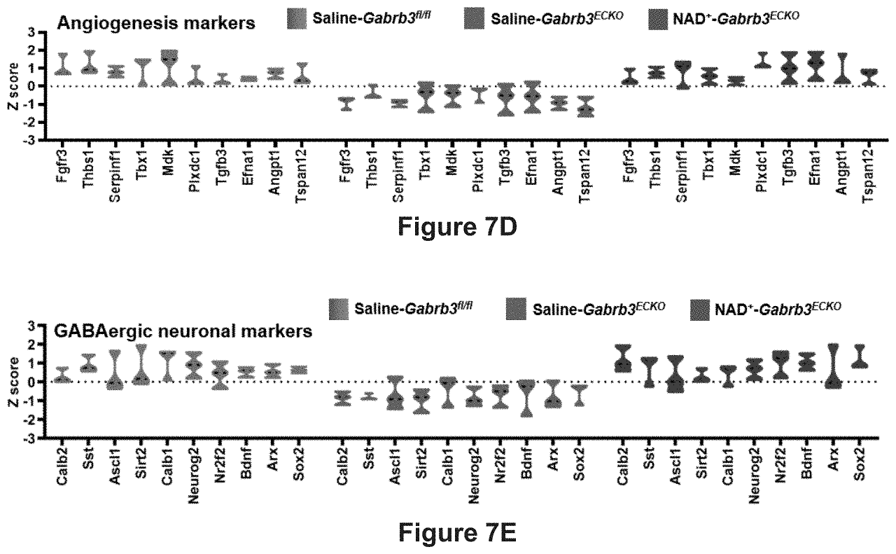

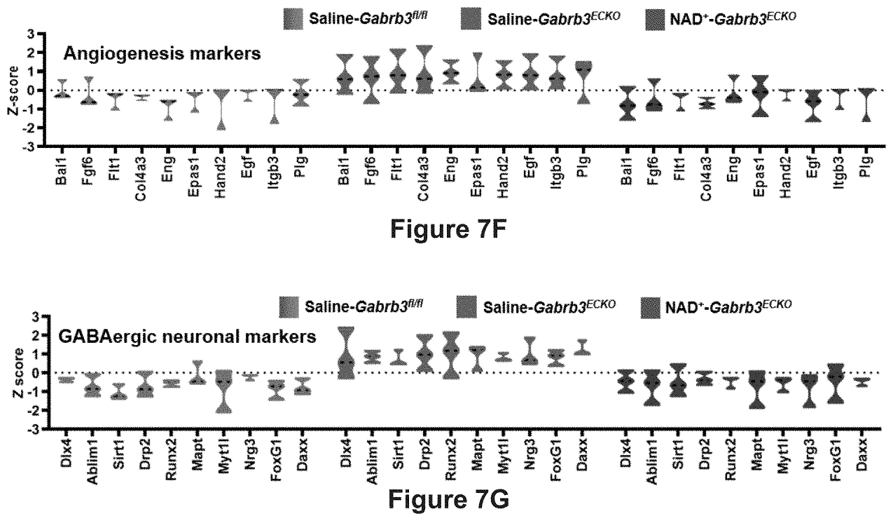

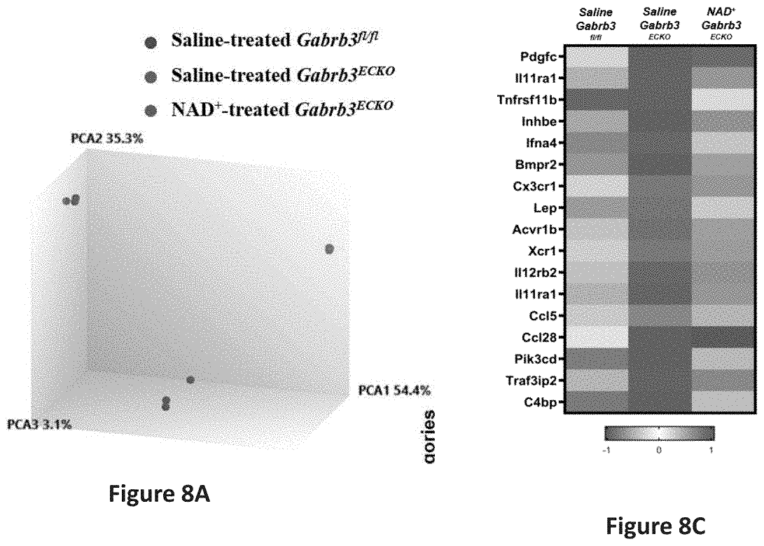

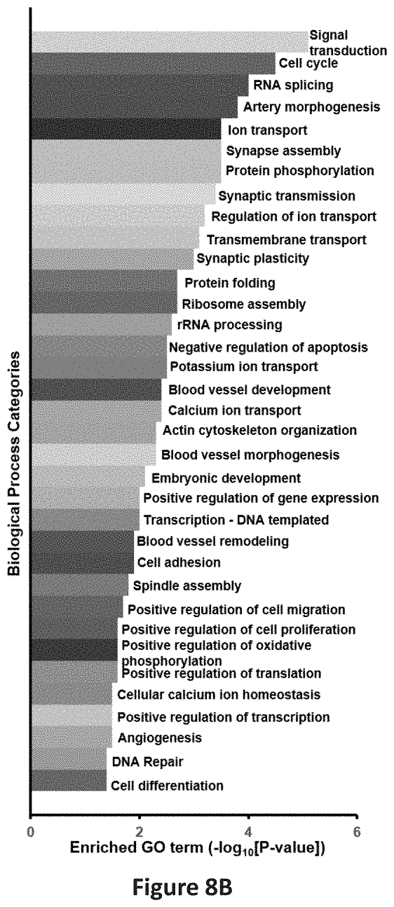

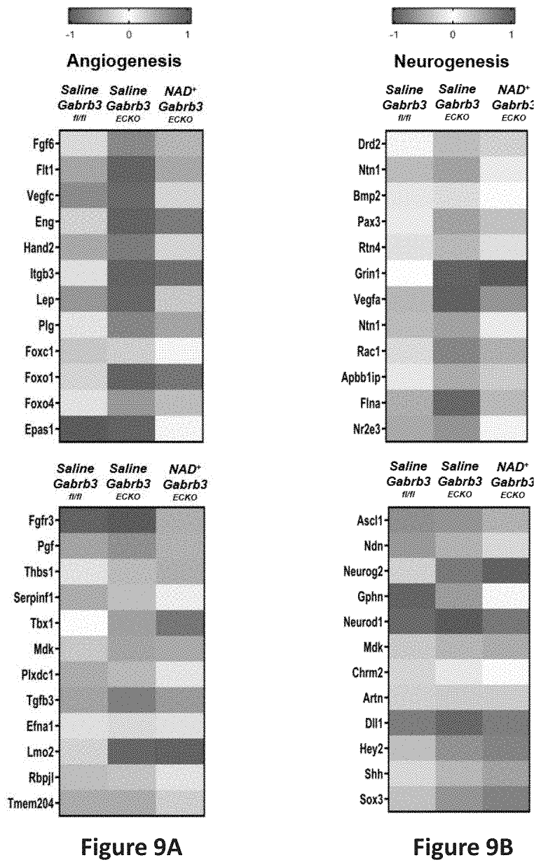

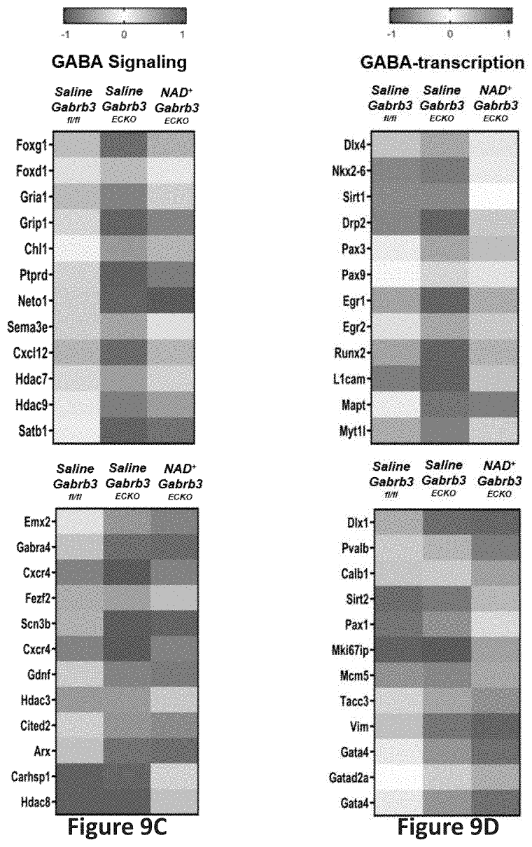

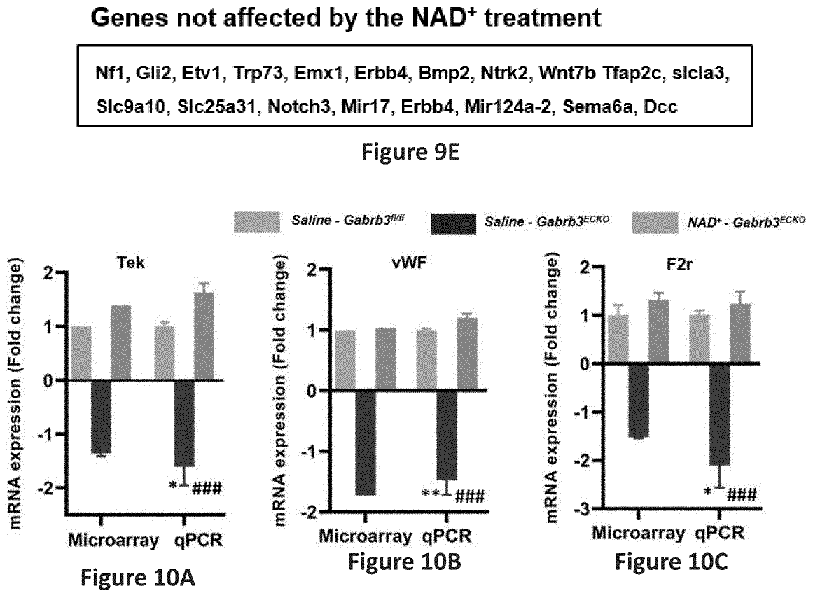

In this exemplary embodiment, to gain deeper insights into NAD⁺ mediated specific actions in the subcortical telencephalon, a micro-dissection of the MGE and striatal tissue from telencephalic slices of saline-treated Gabrb3^{fl/fl} mice, saline-treated Gabrb3^{ECKO} mice, and NAD⁺-treated Gabrb3^{ECKO} mice at E18 was performed. RNA was further extracted, and microarray hybridization on Mouse Gene 2.0 ST arrays (Affymetrix) was performed for Subsequent gene expression analysis (FIG. 7A). Principal component analysis (PCA) plots depicted strict clustering of triplicates from the three samples in the 3D PCA table (FIG. 8A). Differentially expressed genes (fold change cut off ≥+/−50%) in the three groups were compared and represented as heat maps (FIGS. 7B-7C). Interestingly, heat map clusters depicted a shift in the gene expression profile in the NAD⁺-treated Gabrb3^{ECKO} group versus the saline-treated Gabrb3^{ECKO} group and it was similar to the control group for up-or down-regulated gene sets (FIGS. 7B-7C). These results implied that NAD⁺ had the potential to significantly alter gene expression in the embryonic brain. GO Biological process analysis revealed that gene sets in categories such as ‘DNA repair’ ‘homeostasis’, ‘cell cycle’, ‘transcription’, ‘cell differentiation’, ‘angiogenesis’, ‘cell proliferation’, ‘cell adhesion’, ‘blood vessel development’, ‘blood vessel morphogenesis’, ‘positive regulation of cell migration’, ‘calcium in transport’ and several others were comparable between the control and NAD⁺-treated Gabrb3^{ECKO} group (FIG. 8B). Several genes related to inflammation were upregulated in the saline-treated Gabrb3^{ECKO} group, that were restored to normal levels in the NAD⁺ treated group (FIG. 8C). Genes were further classified into specific categories that are essential for embryonic forebrain development: angiogenesis, neurogenesis, GABA signaling and GABA transcription related genes, and the top differentially expressed genes in each category are shown (FIGS. 9A-9E). The gene expression profile revealed that the NAD⁺ treatment had far reaching consequences for critical events during brain development and can modulate signaling events at the level of extracellular receptors, ion channels, transporters, intracellular signaling molecules as well as transcription factors (FIGS. 8A-8C & 9A-9E). Additionally, differential expression of 10 marker genes in angiogenesis and GABAergic neuron categories in saline-treated Gabrb3^{fl/fl}, saline-treated Gabrb3^{ECKO}, and NAD⁺-treated Gabrb3^{ECKO} groups were represented as violin plots (FIGS. 7D-7G). Genes necessary for blood vessel morphogenesis (Fgfr3), homeostatic functions in the regulation of angiogenesis (Thbs1, Serpinf1), vascular sprouting, integrity and survival (Tbx1, Angpt1), mitotic factors (Mdk), vascular cell proliferation and differentiation (Plxdc1, Efna1) and blood-brain barrier development (Tspan12) were significantly downregulated in the saline-treated Gabrb3^{ECKO} group, but was restored to normal levels in the NAD⁺-treated Gabrb3^{ECKO} group (FIG. 7D). Similarly, GABA neuronal subtype related genes (Calb2, Sst, Calb1) and GABAergic neuronal development genes (Sirt2, Ascl1, Nr2f2, Bdnf, Arx and Sox2) that were significantly downregulated in the saline-treated Gabrb3^{ECKO} group were rescued in the NAD⁺-treated Gabrb3^{ECKO} group (FIG. 7E), similar to the saline control group. Additionally, other genes required for vascular development and angiogenesis (Fgf6, Flt1, Hand2, Egf, Itgb3, Plg), hypoxia-related (Epas1) as well as negative regulators of angiogenesis (Bai1, Col4a3, Eng) that were upregulated in the saline-treated Gabrb3^{ECKO} group were restored to normal expression levels in the NAD⁺-treated Gabrb3^{ECKO} group (FIG. 7F). Likewise, several GABAergic pathway regulatory genes (Dlx4, Sirt1, Drp2, Foxg1, Daxx) and axon guidance related genes (Ablim1, Nrg3) that were upregulated in the saline-treated Gabrb3^{ECKO} group were restored to normal expression levels in the NAD⁺-treated Gabrb3^{ECKO} group (FIG. 7G). Collectively, these results indicate that extensive molecular changes occurred in endothelial and neuronal cell types in the Gabrb3^{ECKO} telencephalon that were rescued by the prenatal NAD⁺ treatment.

### Example 4: Mechanistic Insights into NAD⁺ Action on Gabrb3^{ECKO} Endothelial Cells

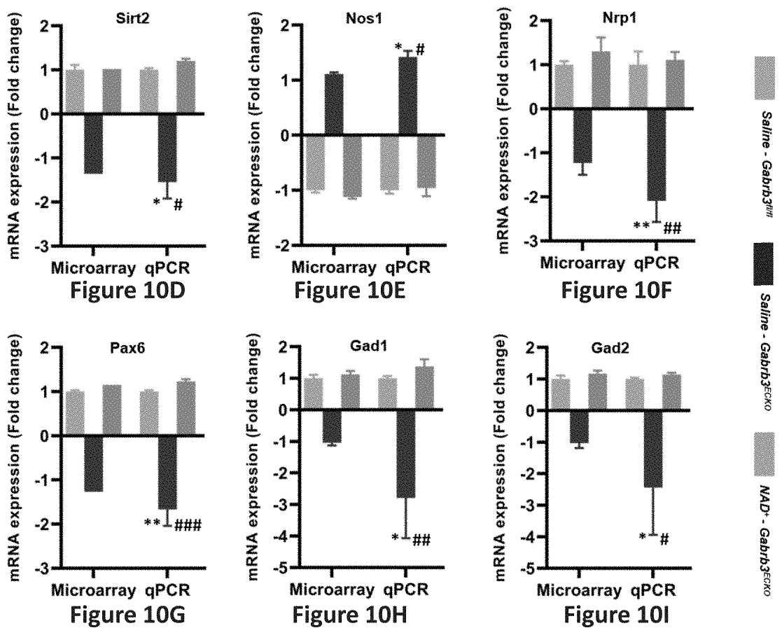

To gain mechanistic insights into NAD⁺ action on endothelial cells, periventricular endothelial cells were isolated from saline-treated Gabrb3^{fl/fl}, saline-treated Gabrb3^{ECKO}, and NAD⁺-treated Gabrb3^{ECKO} groups at the end of the treatment paradigm. These cells were tested for notable changes in gene expression, that were observed in the microarray data, by performing quantitative real-time polymerase chain reaction (qRT-PCR). It was found that the prenatal NAD⁺ treatment had restored the expression of several critical regulators of angiogenesis in Gabrb3^{ECKO} endothelial cells, for instance Tek, vWF, F2r, Sirt2, Nos1 and Pax6 (FIGS. 10A-10G). GABA is synthesized from glutamate by Gad genes. Interestingly, the glutamic acid decarboxylase isoforms, Gad1 and Gad2, were concurrently rescued in Gabrb3^{ECKO} endothelial cells (FIGS. 10H-10I). The qRT-PCR gene expression levels followed a similar trend with that of the levels in the microarray data, reinforcing that the whole-tissue microarray was capable of highlighting some endothelial cells specific gene changes (FIGS. 10A-10I). This data indicates that NAD⁺ is able to directly modulate intracellular GABA levels in Gabrb3^{ECKO} endothelial cells. Taken together, our results implicate NAD⁺ as a critical modulator of angiogenesis in the embryonic forebrain.

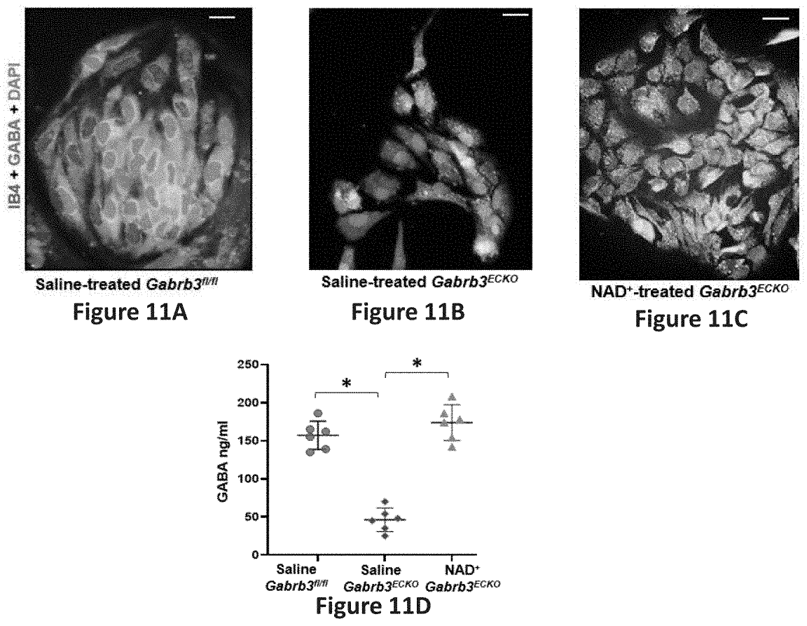

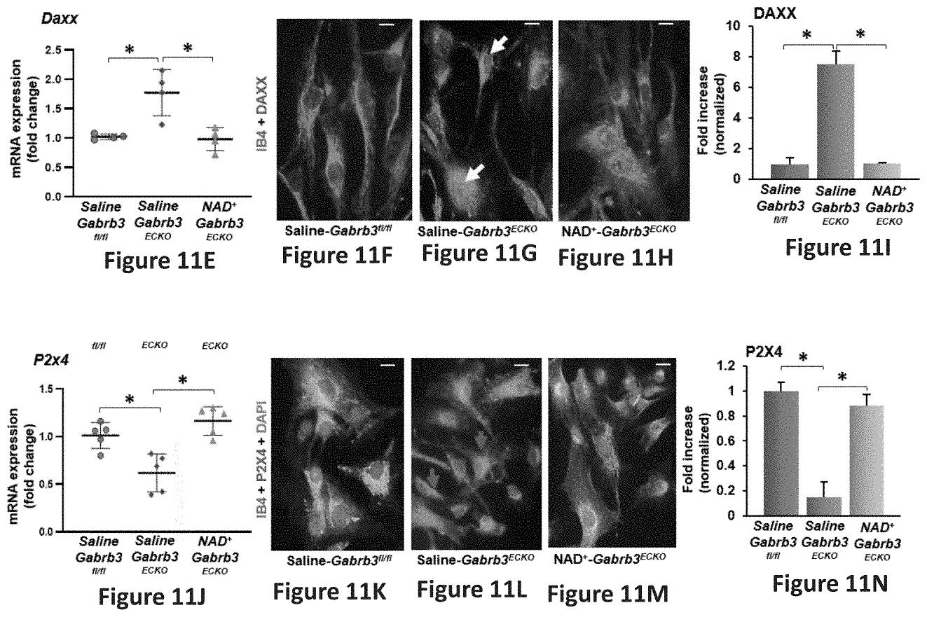

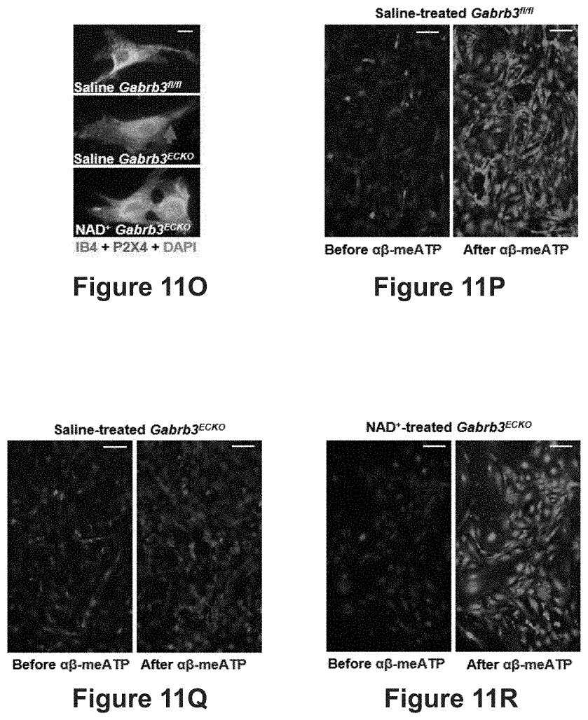

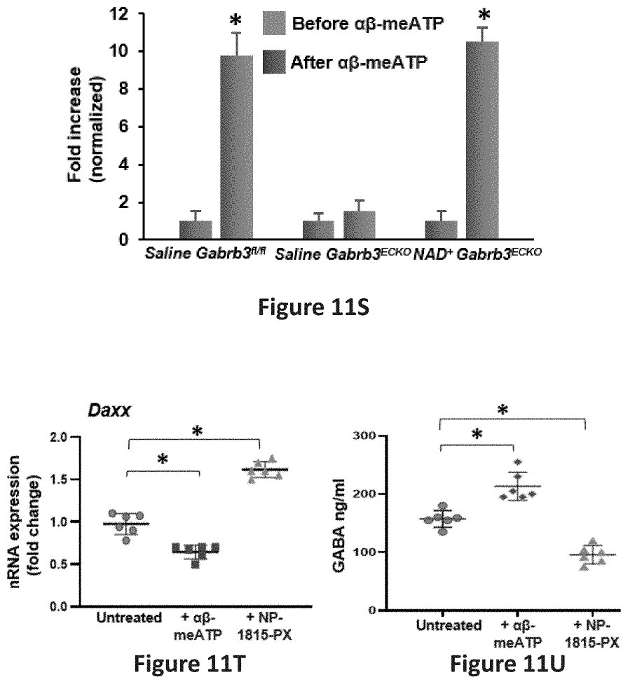

Endothelial cell derived GABA plays dual roles in the embryonic forebrain. It not only activates a positive feedback cycle in endothelial cells that stimulates angiogenesis, but also is an essential chemo-attractive and guidance cue for promotion of long-distance migration of GABAergic interneurons. Endothelial cell specific deletion of Gabrb3 significantly decreased GABA expression and secretion in embryonic periventricular endothelial cells. Since, Gad1 and Gad2 were rescued in Gabrb3^{ECKO} endothelial cells by the prenatal NAD⁺ treatment (FIGS. 10H-10I), it was tested whether GABA expression was rescued in Gabrb3^{ECKO} endothelial cells (FIGS. 11A-11C). Control endothelial cells typically form tight networks or clusters under normal culture conditions. Robust GABA expression was observed by immunohistochemistry in periventricular endothelial cells from the saline control group and these cells also showed robust cluster formation (FIG. 11A). Saline-treated Gabrb3^{ECKO} endothelial cells, in sharp contrast showed a marked reduction in GABA expression and these cells did not form good clusters (FIG. 11B). Prenatal NAD⁺ treatment significantly improved cluster formation in Gabrb3^{ECKO} endothelial cells and GABA expression was also restored (FIG. 4C). Next, we investigated the secretion of GABA by ELISA in the three groups. As expected, there was a significant reduction in GABA secretion from saline-treated Gabrb3^{ECKO} ndothelial cells (FIG. 11D) and this was rescued in NAD⁺-treated Gabrb3^{ECKO} endothelial cells and was similar to the saline control (FIG. 11D). Interestingly, Daxx, a transcriptional repressor of Gad genes that synthesizes GABA (FIGS. 11E-11H), was modulated by the NAD⁺ treatment. Daxx mRNA and protein levels were upregulated in Gabrb3^{ECKO} endothelial cells (FIGS. 11E, 11G, & 11I), as a result of which GABA expression and consequently secretion is affected. NAD⁺ is directly able to regulate Daxx and normalize DAXX levels (FIGS. 11E & 11H-11I), due to which GABA expression (FIG. 11C) and secretion (FIG. 11D) seems to be restored. These results confirmed that the prenatal NAD⁺ treatment can directly modulate synthesis and release of GABA in endothelial cells. Thus, endothelial cell secreted GABA that is essential to promote neuronal migration was rescued in the NAD⁺-treated Gabrb3^{ECKO} group.

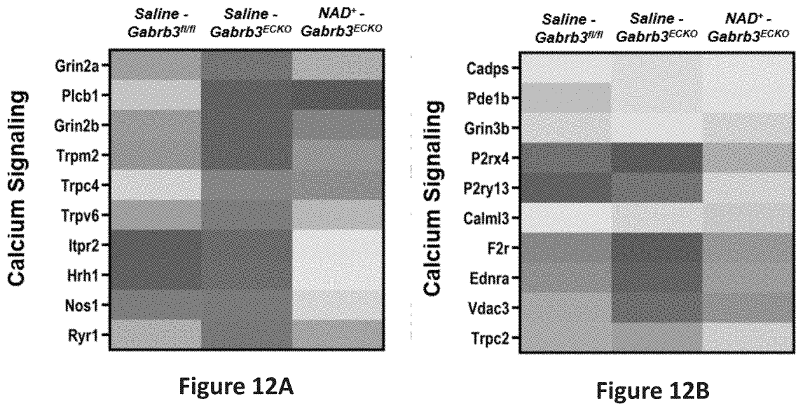

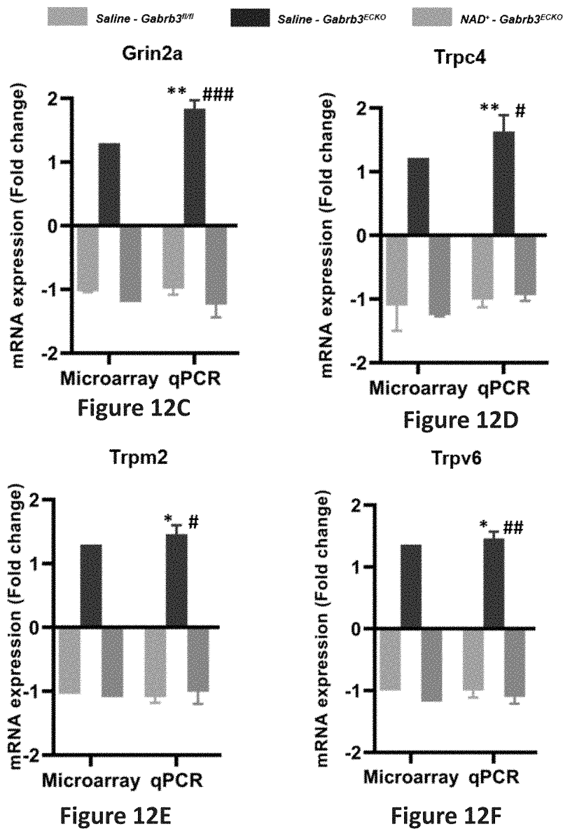

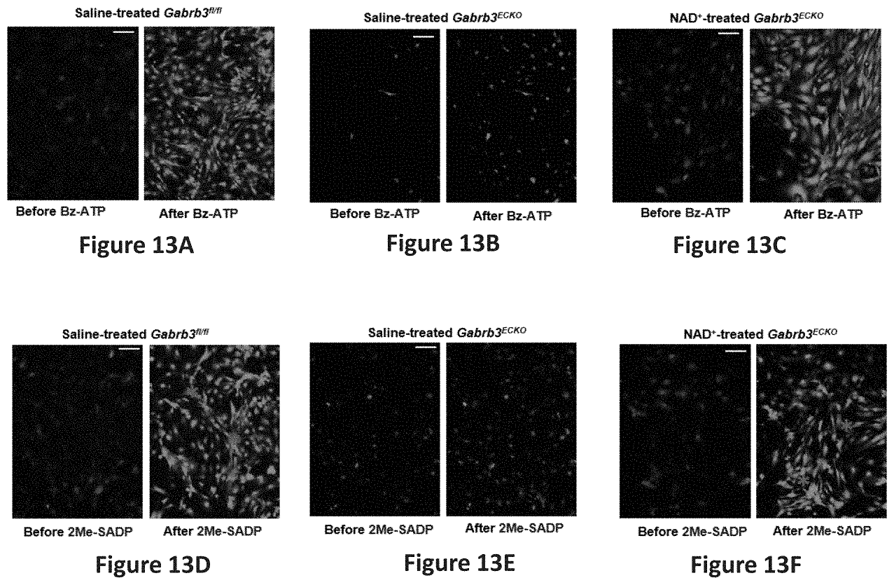

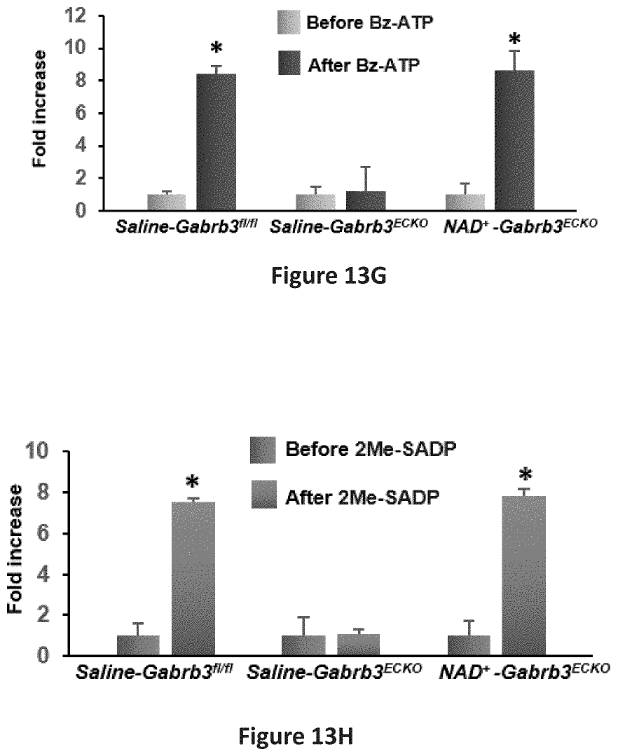

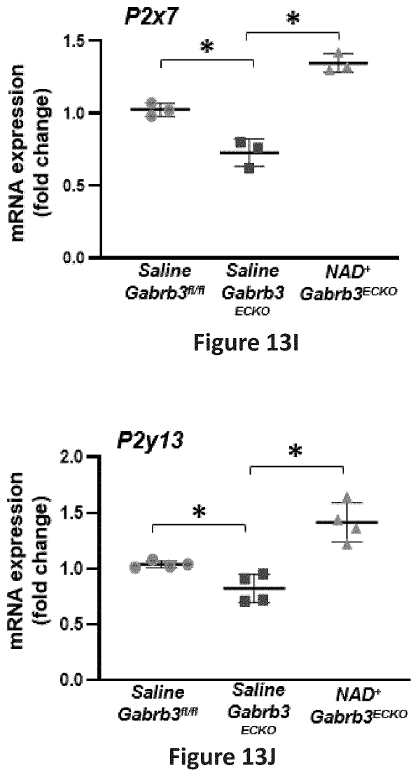

An important aspect that influences endothelial cell proliferation is Ca²⁺ influx, which is important for cell cycle progression in the neocortex. GABA_{A} receptor activation in Gabrb3^{fl/fl} periventricular endothelial cells leads to an influx of Ca²⁺ that influences cell proliferation. However, in Gabrb3^{ECKO} periventricular endothelial cells, due to the deletion of the β3 subunit, the GABA_{A} receptors are dysfunctional. So, the autocrine feedback loop of GABA acting on GABA_{A} receptors will not work in these Gabrb3^{ECKO} endothelial cells to cause Ca²⁺ influx, even if GABA secretion is restored. In the absence of this mechanism, it was questioned whether the prenatal NAD⁺ treatment was able to activate alternate mechanisms to trigger Ca²⁺ influx in Gabrb3^{ECKO} endothelial cells. Interestingly, the gene expression profiling analysis (FIGS. 7A-7G) provided new leads. It was found that calcium signaling related gene expression in the NAD⁺-treated Gabrb3^{ECKO} subcortical telencephalon was similar to the control group for up-or down-regulated gene sets when compared to the saline-treated Gabrb3^{ECKO} group (FIGS. 12A-12B). We validated the rescue of several calcium signaling genes (Grin2a, Trpm2, Trpc4, Trpv6) specifically in NAD⁺-treated Gabrb3^{ECKO} periventricular endothelial cells, and it was comparable to the microarray results (FIGS. 12C-12F). A notable change in purinergic receptor signaling genes in the NAD⁺-treated Gabrb3^{ECKO} group was also observed when compared to the saline-treated Gabrb3^{ECKO} group (FIGS. 12A-12B). Therefore, P2X4 expression at both mRNA and protein levels in periventricular endothelial cells were evaluated, prepared after the six-day saline and NAD⁺ treatment paradigm (FIGS. 11J-11O). P2X4 has been reported as the most abundantly expressed P2X receptor subtype in vascular endothelial cells from several tissues and its deficiency affects normal endothelial cell responses, such as Ca²⁺ influx. However, P2X4 expression and functional significance in embryonic forebrain endothelial cells is unknown. It was found that P2X4 mRNA and protein were robustly expressed in saline-treated Gabrb3^{fl/fl} endothelial cells (FIGS. 11J, 11K, & 11N). Interestingly, P2X4 mRNA and protein expression were significantly decreased in saline-treated Gabrb3^{ECKO} endothelial cells (FIGS. 11J, 11L, & 11N), but its expression was rescued in NAD⁺-treated Gabrb3^{ECKO} endothelial cells (FIGS. 11J, 11M, & 11N). To test whether P2X4 receptor activation on endothelial cells leads to an influx of Ca²⁺, periventricular endothelial cells were incubated from the three groups in the presence of αβ-meATP that has been reported to show agonist activity at P2X receptors, including P2X4 (FIGS. 11N-11Q). Application of αβ-meATP produced a significant increase in intracellular calcium in saline-treated Gabrb3^{fl/fl} endothelial cells and NAD⁺-treated Gabrb3^{ECKO} endothelial cells in calcium imaging assays that were further quantified (FIGS. 11P, 11R, & 11S). However, there was no marked increase in intracellular calcium in saline-treated Gabrb3^{ECKO} endothelial cell proliferation after αβ-meATP application (FIGS. 11Q & 11S). Application of potent agonists for the P2X7 receptor, Bz-ATP, and for the P2Y13 receptor, 2Me-SADP also produced an influx of Ca²⁺ (FIGS. 13A-13H); therefore, multiple purinergic receptor subtypes seem to be activated by NAD⁺. A rescue of P2X7 and P2Y13 mRNA expression was also observed after the NAD⁺ treatment (FIGS. 13I-13J). We next activated or inactivated P2X4 receptors in cultured wild type periventricular endothelial cells, by incubating the endothelial cells in the presence of agonist αβ-meATP or antagonist +NP-1815-PX, followed by testing for Daxx mRNA expression and GABA secretion by ELISA. Activation of purinergic receptors reduced Daxx expression and increased GABA secretion, while inhibition of purinergic receptors increased Daxx expression and reduced GABA secretion (FIGS. 11T-11U). These results indicate an inverse correlation between Daxx expression and GABA secretion that is modulated directly by purinergic receptor signaling. Together, these results indicate that purinergic receptor signaling is restored in Gabrb3^{ECKO} periventricular endothelial cells after NAD⁺ treatment and elucidates an alternate mechanism for NAD⁺ mediated rescue of endothelial cell proliferation and angiogenesis in the prenatal developmental period.

### Example 5: Prenatal NAD⁺ Treatment Rescued Adult Brain Blood Flow and Ameliorated Abnormal Behaviors in the Gabrb3^{ECKO} Mice

The consequences of loss of endothelial Gabrb3 in the embryonic brain persisted in the adult brain, reflecting as reduced vascular densities and functional changes in blood vessels as well as a reduction of cortical interneurons. This resulted in multifaceted behavioral deficits which are common to several different psychiatric diseases; with symptoms that included impaired reciprocal social interactions, communication deficits and heightened anxiety. Since the prenatal NAD⁺ treatment mediated rescue of cellular and molecular aspects of the Gabrb3^{ECKO} embryonic brain (FIGS. 4A-40, 6A-6Y, 7A-7G, & 11A-11U), and restored the vascular and GABAergic neuronal deficits in the adult cerebral cortex (FIGS. 6U-6Y), another experiment questioned whether it could contribute to a rescue of blood flow and amelioration of abnormal behaviors.

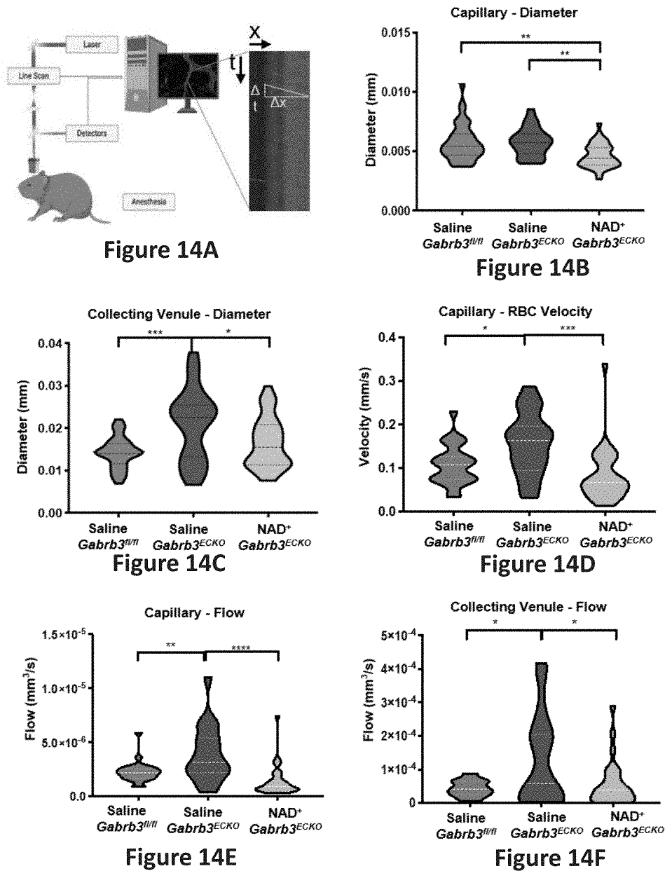

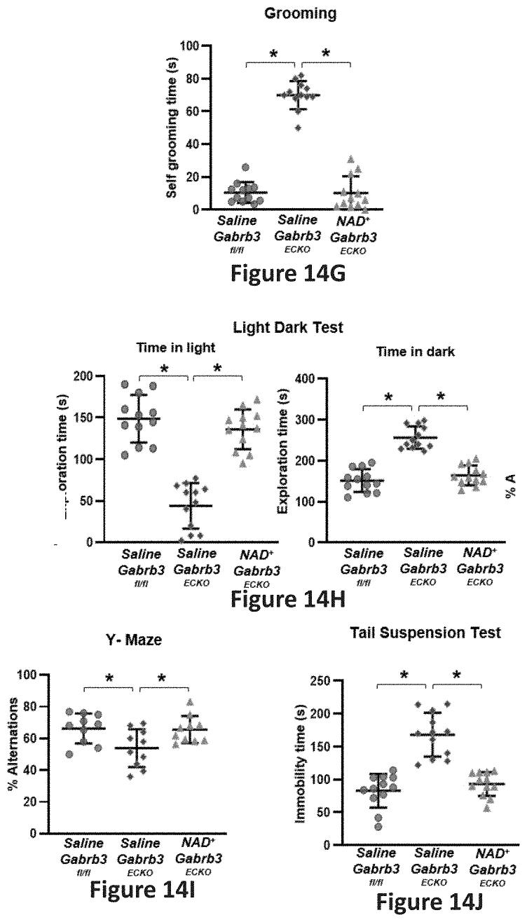

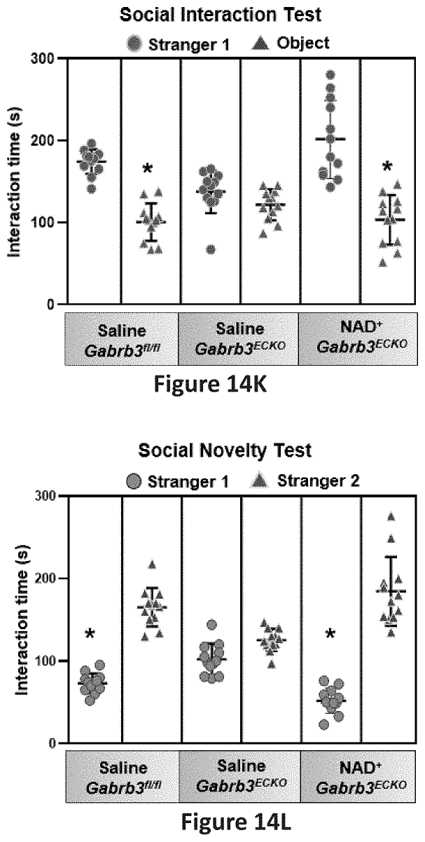

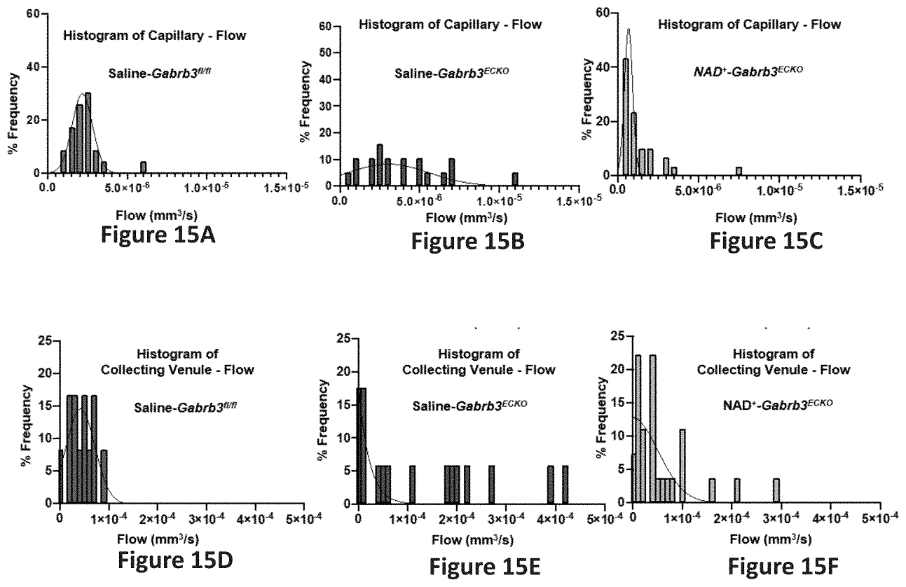

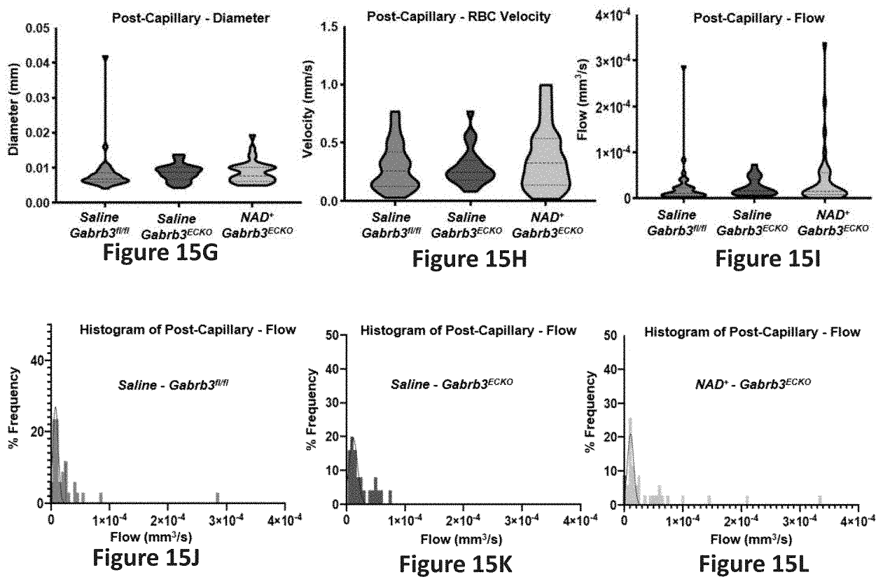

Therefore, vessel diameters, red blood cell (RBC) velocity, and blood flow were evaluated in the cerebral cortex of saline-treated Gabrb3^{fl/fl} mice, saline-treated Gabrb3^{ECKO} mice, and NAD⁺-treated Gabrb3^{ECKO} mice (FIGS. 14A-14F) using multiphoton laser-scanning microscopy (MPLSM) and a sophisticated cranial window model. An increase in the diameter of capillaries and collecting venules was observed in the saline-treated Gabrb3^{ECKO} mice compared to saline-treated Gabrb3^{fl/fl} mice (FIGS. 14B-14C). Interestingly, this morphological alteration was rescued in NAD⁺-treated Gabrb3^{ECKO} mice (FIGS. 14B-14C). Similarly, RBC velocity was significantly increased in capillaries in saline treated-Gabrb3^{ECKO} mice when compared to saline-treated Gabrb3^{fl/fl} mice and this was reversed in NAD⁺-treated Gabrb3^{ECKO} mice (FIG. 14D). Consistently, blood flow in capillaries and collecting venules was also significantly reduced in NAD⁺-treated Gabrb3^{ECKO} mice when compared to saline-treated Gabrb3^{ECKO} mice (FIGS. 14E-14F). Histograms depict the changes in blood flow distribution in capillaries and collecting venules between the three groups (FIGS. 15A-15F). No significant changes were observed in vessel diameter, RBC velocity or blood flow in the post-capillary venules of saline-treated Gabrb3^{ECKO} mice when compared to controls (FIGS. 15G-15L). These results indicate that the capillaries and the collecting venules were the most significantly affected units of the microcirculation in saline treated-Gabrb3^{ECKO} mice; that were rescued by the prenatal NAD⁺treatment.

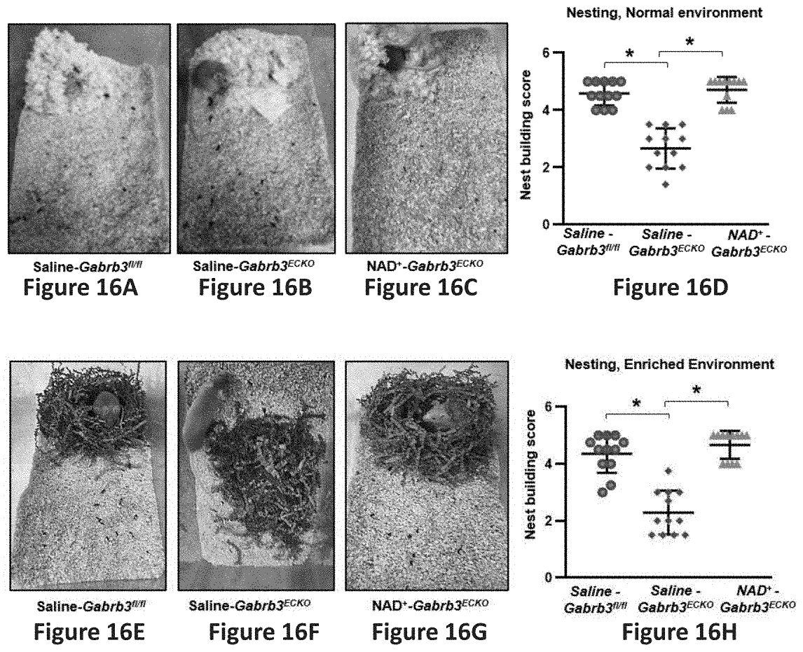

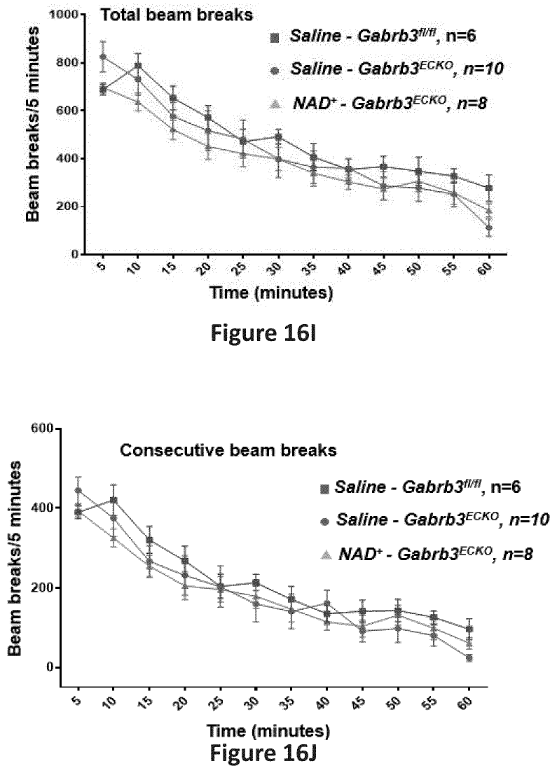

The prenatal NAD⁺ treatment did not have any effect on litter size and pups grew normally to adulthood. Therefore, behavioral tests were performed to screen for stress, anxiety, locomotion, cognition, and sociability in saline-treated Gabrb3^{fl/fl} mice, saline-treated Gabrb3^{ECKO} mice and NAD⁺-treated Gabrb3^{ECKO} mice. Mice from saline and NAD⁺-treated groups were housed individually in cages containing wood chip bedding and two nestlets (pressed cotton) (FIGS. 16A-16D) or more naturalistic material like shredded paper strips (FIGS. 16E-16H). Saline-treated Gabrb3^{ECKO} mice showed poor nest building behavior in both normal (FIGS. 16B & 16D), and enriched (FIGS. 16F & 16H) environments when compared to saline-treated Gabrb3^{fl/fl} mice (FIGS. 16A, 16D, 16E, & 16H), indicative of heightened stress/anxiety and impaired home cage social behavior. NAD⁺-treated Gabrb3^{ECKO} mice showed significant improvement in their nest building ability and were comparable to saline control mice (FIGS. 16C, 16D, 16G, & 16H). This rescue of home cage social behavior was a robust indication of well-being in NAD⁺-treated Gabrb3^{ECKO} mice. The severe grooming behavior indicative of impaired home-cage social behavior and increased stress/anxiety in saline-treated Gabrb3^{ECKO} mice was also ameliorated after the prenatal NAD⁺ treatment (FIG. 14G).

Anxiety was next assessed with the classic light-dark transition test which triggers a struggle between the desires to explore a novel environment versus natural aversion of a brightly illuminated open space. While the saline-treated Gabrb3^{ECKO} mice showed an aversion to brightly lit open space and preferred the dark area (FIG. 14H), both saline-treated Gabrb3^{fl/fl} mice and NAD⁺-treated Gabrb3^{ECKO} mice made several entries into the brightened space and spent equivalent times between the light and dark sides of the open field (FIG. 5H). Open field locomotor activity defined as breaking of total or consecutive infrared photobeams was monitored for 60 minutes, and no significant differences were observed between the three groups (FIGS. 16I-16J). The Y maze spontaneous alternation test was used to evaluate for spatial learning and memory in the three groups of mice. After introduction to the center of the maze, the mice were given free access to all three arms. If a different arm is chosen by the mouse than the one it arrived from, this choice is called an alteration and is considered the correct response. The total number of arm entries and the sequence of entries are recorded in order to calculate the percentage of alternation. Saline-treated Gabrb3^{fl/fl} mice and NAD⁺-treated Gabrb3^{ECKO} mice showed a significant increase in percentage alternations when compared to saline-treated Gabrb3^{ECKO} mice, indicative of an improvement in cognition (FIG. 14I). Next, we used the tail suspension test to evaluate the mice for depressive behavior. When suspended by their tails, normal mice will struggle to face upward and show apparent escape efforts that include running movements, body torsion, reaching and shaking. Immobility of the mouse is defined as a depressive state when the mouse has given up and doesn't want to put in the effort to try to escape. Saline-treated Gabrb3^{ECKO} mice showed longer periods of immobility compared to saline control mice (FIG. 14J). NAD⁺-treated Gabrb3^{ECKO} mice had significantly lower immobility times, similar to saline controls (FIG. 14J).

NAD⁺-treated Gabrb3^{ECKO} mice also showed a significant improvement in social communication skills. In a three-chamber social communication test, saline-treated Gabrb3^{ECKO} mice showed no preference for a stranger mouse and spent an approximately similar time in investigating the stranger mouse versus an inanimate object signifying impaired sociability. In contrast, NAD⁺-treated Gabrb3^{ECKO} mice interacted with the stranger mouse for a significantly longer duration than with the inanimate object, similar to saline controls (FIG. 14K). In the social novelty phase, when a new stranger mouse was introduced into the previously empty cylinder, both saline-treated Gabrb3^{fl/fl} mice and NAD⁺-treated Gabrb3^{ECKO} mice showed a marked preference for stranger 2 versus the now familiar stranger 1, while Gabrb3^{ECKO} mice did not show such a preference. This is indicative of rescue of social motivation, memory and novelty exploration in NAD⁺-treated Gabrb3^{ECKO} mice (FIG. 14L). Collectively, these results provide novel evidence that NAD⁺ treatment during a select prenatal developmental window is sufficient to rescue blood flow in an irreversible manner and ameliorate abnormal behaviors.

### Example 6: Testing the Effect of NAD⁺ and GABA on Embryonic Forebrain Endothelial Cells

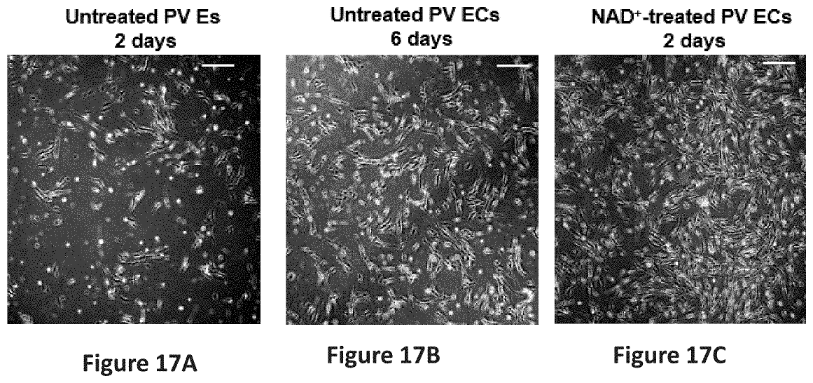

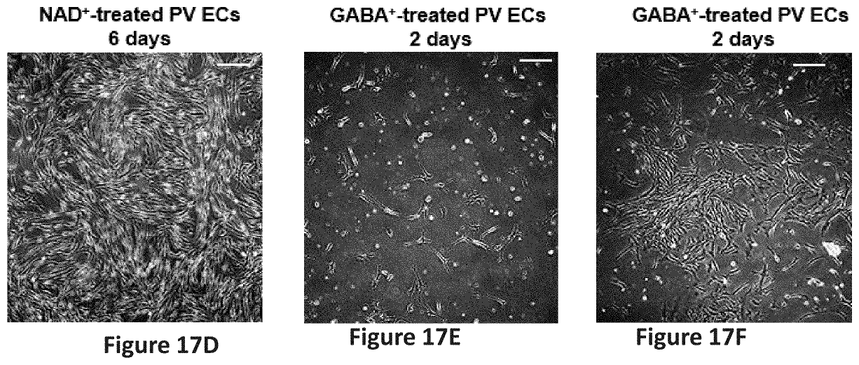

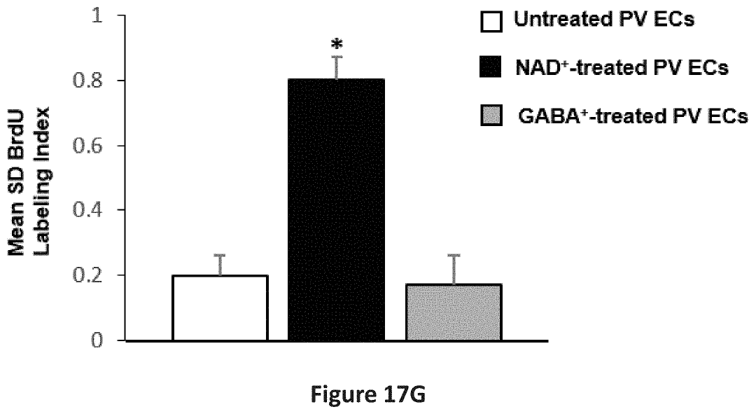

Effects of NAD⁺ addition to periventricular endothelial cells isolated from E15 wildtype (CD1) forebrain was more robust than GABA. (FIGS. 17A-17F) Phase contrast images of periventricular endothelial cells cultured from E15 CD1 forebrain at different time points in untreated conditions (FIGS. 17A-17B) or after plating with NAD⁺ addition (100 μM; FIGS. 17C-17D) or GABA addition (5 μM; FIGS. 17E-17F). Robust proliferation was observed only after NAD⁺ addition (red asterisks, FIGS. 17C-17D). Different concentrations of GABA (30 μM and 100 μM) were also tried, but there was no notable effect on cell proliferation similar to the NAD⁺ group. (FIG. 17G) All groups of periventricular endothelial cells were exposed to BrdU (1 mM BrdU per ml medium) for 1 hour followed by Isolectin B4/BrdU double labeling. Quantification of BrdU labeling indices; Data represents mean±SD (n=8, *P<0.05; Student's t-test).

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A method for preventing a psychiatric disorder, comprising:
providing a therapeutically effective amount of an angiogenesis pathway regulator to an individual.

2. The method of claim 1, wherein the angiogenesis pathway regulator can cross a utero-placental barrier.

3. The method of claim 1, wherein the angiogenesis pathway regulator is selected from the group consisting of NAD⁺, GABA, VEGF, and FGF.

4. The method of claim 1, wherein the angiogenesis pathway regulator is NAD⁺.

5. The method of claim 4, wherein NAD⁺ is administered at a dose of between 10 mg/kg to 40 mg/kg.

6. The method of claim 1, wherein the administering step is performed orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, or perispinally.

7. The method of claim 1, wherein the administering step is performed intraperitoneally.

8. The method of claim 1, wherein the individual is pregnant.

9. The method of claim 8, wherein the offspring of the pregnant individual is susceptible to a psychiatric disorder.

10. The method of claim 9, wherein the psychiatric disorder is selected from the group consisting of autism, epilepsy, schizophrenia, OCD, anxiety, and depression.

11. The method of claim 1, further comprising identifying the individual to be treated.

12. The method of claim 11, wherein identifying the individual individual to be treated comprises identifying a neurological malformation in the individual.

13. The method of claim 12, wherein the neurological malformation is identified by a CT scan or MRI.

14. The method of claim 11, wherein the individual is identified by measuring NAD⁺ levels in the individual.

15. A pharmaceutical formulation for the prevention of a psychiatric disorder, comprising a therapeutically effective amount of an angiogenesis pathway regulator.

16. The pharmaceutical formulation of claim 15, wherein the angiogenesis pathway regulator is selected from the group consisting of NAD⁺, GABA, VEGF, and FGF.

17. The pharmaceutical formulation of claim 15, wherein the angiogenesis pathway regulator can cross a utero-placental barrier.

18. The pharmaceutical formulation of claim 15, wherein the angiogenesis pathway regulator is NAD⁺.

19. The pharmaceutical formulation of claim 18, wherein NAD⁺ is at a dose of between 10 mg to 40 mg.

20. The pharmaceutical formulation of claim 15, wherein NAD⁺ is at a dose of 10 mg in 100 μL of saline.

21. The pharmaceutical formulation of claim 15, further comprising at least one of the following: a buffer, a stabilizer, a balancer, a flavor, a filler, a disintegrant, a lubricant, a glidant, or a binder.

22. The pharmaceutical formulation of claim 15, wherein the angiogenesis pathway regulator is formulated for administration orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, or perispinally.

23. The pharmaceutical formulation of claim 15, wherein the angiogenesis pathway regulator is NAD⁺ and is formulated for intraperitoneal administration.
